(19) United States
(12) Patent Application Publication
(10) Pub. No.: US 2012/0052579 A1
(43) Pub. Date: Mar. 1, 2012

(54) PEPTIDE-MODIFIED MICROCARRIERS FOR CELL CULTURE

(76) Inventors: Simon Kelly Shannon, Horseheads, NY (US); Florence Verrier, Corning, NY (US)

(21) Appl. No.: 13/193,073

(22) Filed: Jul. 28, 2011

Related U.S. Application Data
(60) Provisional application No. 61/377,722, filed on Aug. 27, 2010.

(51) Int. Cl. C12N 5/071; C12N 5/0735; C08F 222/06; C12N 5/0797

(52) U.S. Cl. 435/396; 525/54.1

(57) ABSTRACT

A cell culture article including a microcarrier having a peptide-modified polymer surface of the formula (I) where AAj represents at least one covalently bonded peptide, j is an integer of from 5 to 50, m, n, o, Sur, X, R, R′, and the mer ratio (m-o:n:o), including salts thereof, are as defined herein. Also disclosed are methods for making and using the cell culture article, as defined herein.

### CROSS-REFERENCE TO RELATED APPLICATIONS

This application claims the benefit of priority under 35 U.S.C. §119 of U.S. Provisional Application Ser. No. 61/377,722, filed on Aug. 27, 2010, the content of which is relied upon and incorporated herein by reference in its entirety.

### BACKGROUND

The disclosure generally relates to surface-modified microcarriers having surfaces adapted for cell culture applications.

### SUMMARY

The disclosure provides biologically-compatible, peptide-modified microcarriers for cell culture, cell culture articles incorporating the peptide-modified microcarriers, and methods for making and using the cell culture articles.

### BRIEF DESCRIPTION OF THE DRAWING(S)

In embodiments of the disclosure:

FIG. 1 schematically shows a process used to prepare cell culture surfaces including modifying a reactive polymer surface with biologically-compatible peptide sequences.

FIG. 2 is a schematic drawing of a cross-section of an exemplary coated microcarrier.

FIG. 3 is a schematic drawing of a cross-section of an exemplary coated microcarrier with a conjugated polypeptide.

FIG. 4 is a flow diagram of an exemplary method of forming a coated microcarrier.

FIG. 5 is a reaction scheme of an exemplary method for forming a coated microcarrier.

FIG. 6 is a reaction scheme of an exemplary method for forming a coated microcarrier.

FIG. 7 is a reaction scheme of an exemplary method of regenerating a coated microsphere.

FIG. 8 shows scanning electron micrographs of as received low density glass microcarriers (A1, B1 and C1), and low density glass microcarriers coated with anhydride polymer (A2, B2 and C2) at various magnifications.

FIG. 9 shows crystal violet blue staining of uncoated and coated hydrolyzed maleic anhydride coated polystyrene (top) and glass (bottom) microspheres.

FIG. 10 shows fluorescence images of rhodamine labeled vitronectin-conjugated microspheres at various concentration levels.

FIG. 11 shows a graph of the relationship between BCA estimated peptide density and VN challenge concentration.

FIG. 12 is a confocal microscope image of a polystyrene microcarrier with fluorescently labeled coating.

FIG. 13 shows HT1080 human fibrosarcoma cell adhesion on coated microcarriers.

FIG. 14 shows HT1080 human fibrosarcoma cell adhesion on coated microcarriers.

FIG. 15 shows a microscopy images illustrating BG01V/hOG human embryonic stem cell growth on vitronectin peptide-modified glass microcarriers 5 days after seeding. A (brightfield image), B (fluorescence, FITC).

FIG. 16 shows a graph of quantification of BG01V/hOG human embryonic stem cells after 2 days and 5 days of culture performed on vitronectin peptide-modified glass microcarriers, and on Matrigel coated beads (Matrigel™ CM), and Cytodex™ 3 as comparative examples.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not limiting and merely set forth some of the many possible embodiments of the claimed invention.

### DEFINITIONS

“Peptide” means a sequence of amino acids that can be chemically synthesized or can be recombinantly derived, but that are not isolated as entire proteins from animal sources. For the purposes of this disclosure, peptides and peptides are not whole proteins. Peptides and peptides can include amino acid sequences that are fragments of proteins. For example peptides and peptides can include sequences known as cell adhesion sequences such as RGD. Peptides can be of any suitable length, such as between three and 30 amino acids in length. Peptides can be acetylated (e.g. Ac-LysGlyGly) or amidated (e.g. SerLysSer-NH₂) to protect them from being broken down by, for example, exopeptidases. Peptide sequences are referred to herein by their one letter amino acid codes and by their three letter amino acid codes. These codes can be used. It will be understood that these modifications are contemplated when a sequence is disclosed.

“dEMA,” “derivatized EMA,” “derivatized ethylene-maleic anhydride copolymer,” or like terms refer to an EMA polymer which has been pre-blocked with at least one of various exemplary agents, such as ethanol amine or methoxyethyl amine, see commonly owned and assigned U.S. Pat. No. 7,781,203.

“Microcarrier,” “microsphere,” “microbead,” “bead,” or like terms mean a small discrete particle for use in culturing cells and to which cells can attach. Microcarriers can be in any suitable shape, such as rods, spheres, and the like. In embodiments, a microcarrier can include a microcarrier base that is coated to provide a surface suitable for cell culture. A peptide can be bonded, grafted, or otherwise attached to the surface coating.

“Include,” “includes,” or like terms means encompassing but not limited to, that is, inclusive and not exclusive.

“About” modifying, for example, the quantity of an ingredient in a composition, concentrations, volumes, process temperature, process time, yields, flow rates, pressures, and like values, and ranges thereof, employed in describing the embodiments of the disclosure, refers to variation in the numerical quantity that can occur, for example: through typical measuring and handling procedures used for making compounds, compositions, composites, concentrates or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture. The claims appended hereto include equivalents of these “about” quantities.

“Consisting essentially of” in embodiments refers, for example, to a coated microcarrier composition, to a method of making or using the coated microcarrier composition, or formulation, and articles, devices, or any apparatus of the disclosure, and can include the components or steps listed in the claim, plus other components or steps that do not materially affect the basic and novel properties of the compositions, articles, apparatus, or methods of making and use of the disclosure, such as particular reactants, particular additives or ingredients, a particular agents, a particular surface modifier or condition, or like structure, material, or process variable selected. Items that can materially affect the basic properties of the components or steps of the disclosure or that can impart undesirable characteristics to the present disclosure include, for example, cell culture media which cannot provide exemplary growth and differentiation of selected cells or their progenitors. “Consisting essentially of,” “consisting of,” and like phases are subsumed in “comprising”. Accordingly, a microcarrier comprising a microcarrier base and a coating includes a microcarrier consisting essentially of, or consisting of, a microcarrier base and a coating.

The indefinite article “a” or “an” and its corresponding definite article “the” as used herein means at least one, or one or more, unless specified otherwise.

“Have”, “having”, “include”, “including”, “comprise”, “comprising” or like terms are used in their open ended sense, and generally mean “including, but not limited to”.

Abbreviations, which are well known to one of ordinary skill in the art, can be used (e.g., “h” or “hr” for hour or hours, “g” or “gm” for gram(s), “mL” for milliliters, and “rt” for room temperature, “nm” for nanometers, and like abbreviations). All scientific and technical terms used herein have meanings commonly used in the art unless otherwise specified. The definitions provided herein are to facilitate understanding of certain terms used frequently herein and are not meant to limit the scope of the present disclosure.

Other abbreviation, such as the alphabet of single letter or three letter representations for an amino acid or combinations thereof for a peptide sequence are readily apparent and can be found, for example, in Lehninger, Principles of Biochemistry, 5th Ed., © 2009.

Specific and preferred values disclosed for components, ingredients, additives, and like aspects, and ranges thereof, are for illustration only; they do not exclude other defined values or other values within defined ranges. The compositions, apparatus, and methods of the disclosure can include any value or any combination of the values, specific values, more specific values, and preferred values described herein.

The attachment, proliferation and expansion of cells that have the ability to self-renew (e.g., stem cells) in a scalable fashion are important if cells were to be used for therapeutic purposes. Microcarriers are high-surface area functionalized microspheres, typically 100 to 500 microns in diameter, used for suspension culture and expansion of anchorage dependent cells. Microcarriers are typically stirred or agitated in cell culture media and provide a very large attachment and growth surface area to volume ratio relative to more traditional culture equipment. Due to the high surface to volume ratio, microcarriers enable the production of a large number of cells in small volumes, which lead to increased cell culture yields, improved mass transport of nutrients, reduction of equipment size, volume and cost of culture (Bryan, G. In: Masters, J. R. W., ed. Animal cell culture; a practical approach. New York; Oxford University Press; 2000. 19-66; Justice, B. A., et. al., Drug Disc. Today. 2009, 14, 102-107).

Many of the commercially available microcarriers provide non-specific attachment of cells to the carriers for cell growth (see e.g., US 2008/0199959; and Phillips, B. W., et. al., J. Biotech. 2008, 138, 24-32). While useful, such microcarriers do not allow for biospecific cell adhesion which can lead to cells that are difficult to maintain in a particular state (e.g., undifferentiated state) and thus do not readily allow for tailoring of characteristics of the cultured cells. For example, due to non-specific interactions it can be difficult to maintain cells, such as human embryonic stem cells (hESCs), in a particular state of differentiation or to direct cells to differentiate in a particular manner.

Some currently available surfaces provide for bio-specific adhesion, but employ animal derived coatings such as collagen, laminin or gelatin and other animal derived components. Such animal derived coatings can expose the cells to potentially harmful viruses or other infectious agents which could be transferred to patients if the cells are used for a therapeutic purpose. In addition, such viruses or other infectious agents can compromise general culture and maintenance of the cultured cells. Further, such biological products tend to be vulnerable to batch variation and limited shelf-life.

Extra-cellular matrix proteins derived from animals can introduce infective agents such as viruses or prions. These infective agents can be taken up by cells in culture and, upon the transplantation of these cells into a patient, can be taken up into the patient. Therefore, the addition of these factors in or on cell culture surfaces can introduce new disease even as they address an existing condition. In addition, these animal-derived additives or cell surface coatings can lead to significant manufacturing expense and lot-to-lot variability.

Some synthetic, chemically-defined surfaces have been shown to be effective in culturing cells, such as embryonic stem cells, in chemically defined media. However, the ability of such surfaces to support culture on microcarriers has not yet been realized nor has methods for applying such surfaces to microcarriers.

The present disclosure provides microcarriers for culturing cells. In embodiments, the microcarriers can configured to support, for example, proliferation and maintenance of undifferentiated stem cells in chemically defined media.

In embodiments, the disclosure provides a method for making a maleic anhydride copolymer synthetic peptide microcarrier surface, for example dEMA having RGD peptide sequences derived from vitronectin and bone sialoprotein for suspension culture of human embryonic stem cells. An RGD peptide sequence, that is arginylglycylaspartic acid, is a tripeptide composed of L-arginine, glycine, and L-aspartic acid. The sequence is a common element in cellular recognition.

In embodiments, the peptide modified microcarriers are formed by (i) coating with a tie layer onto the base microcarrier; (ii) coating the maleic anhydride polymer onto the base microcarrier; (iii) conjugating the cell-adhesive peptide to the maleic anhydride coated microcarrier directly (by way amine/anhydride reaction) or indirectly (by way of carboxylate activation chemistry) through amide bond formation.

In embodiments, a method to regenerate hydrolyzed (i.e., otherwise un-reactive) maleic anhydride coated microcarriers for re-use as a support for direct peptide conjugation. The regenerated maleic anhydride coating enables a method to directly bind adhesion peptide sequences (for example, RGD peptides) to prepare synthetic peptide-derived microcarrier supports. Commonly owned and assigned U.S. Pat. No. 7,781,203, issued Aug. 24, 2010, to Frutos, et al., mentions supports based on, for example, poly(ethylene-alt-maleic anhydride) (EMA) derived surfaces for assaying analytes and methods of making and using thereof.

One or more of the various embodiments presented herein provide one or more advantages over prior articles and systems for culturing cells. For example, peptide-modified microcarriers described herein have been shown to support cell adhesion without the need of animal derived biocoating which limits the risk of pathogen contamination. This is especially relevant when cells are dedicated to cell therapies. The methods described herein allow for the preparation of surfaces having a wide range of adhesive properties based on the peptide origin (e.g., bone sialoprotein or vitronectin). Such microcarriers can also be advantageously used for culturing cells other than stem cells when animal derived products such as collagen, gelatin, fibronectin, etc., are undesired or prohibited. These and other advantages will be readily understood from the following detailed descriptions when read in conjunction with the accompanying drawing.

### 1. Surface Modified Microcarrier

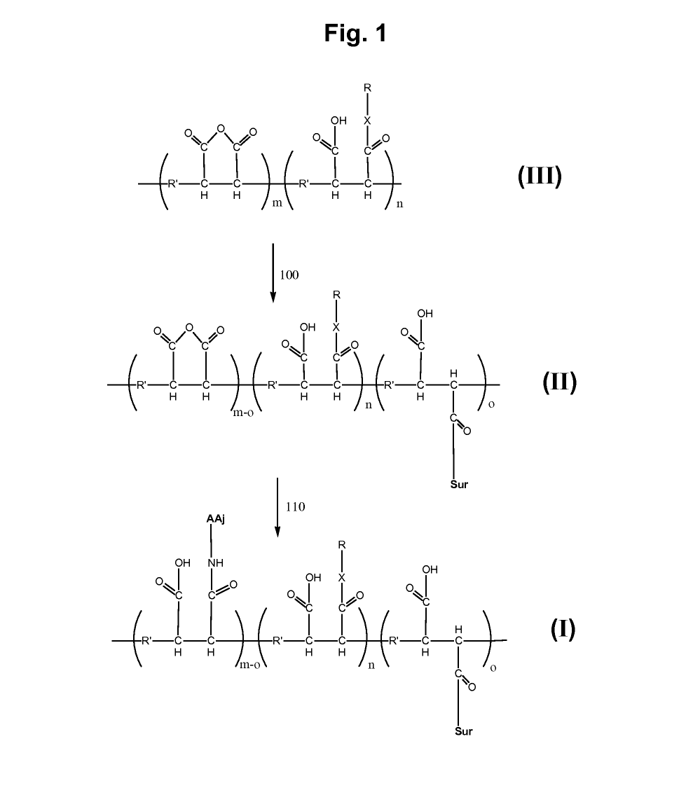

Referring to FIG. 1, the peptide modified surface includes a microcarrier surface (Sur), a derivatized anhydride polymer coating, and peptide (AAj). The derivatized anhydride surface coating with or without peptide conjugation provide a microcarrier surface to which cells can attach for the purposes of cell culture. In various embodiments, the dEMA coating layer is deposited on or formed on a microcarrier surface of an intermediate layer that is associated with the base microcarrier surface (Sur) via covalent or non-covalent interactions, either directly or via one or more additional intermediate layers (not shown). In such cases, the intermediate is considered, for the purposes of this disclosure, to be a part of the microcarrier base.

In embodiments, this disclosure provides a peptide grafted maleic anhydride copolymer coated on a microcarrier surface, for example, (i) derivatized ethylene-maleic anhydride copolymer (dEMA) having RGD peptide sequences derived from vitronectin, laminin, and bone sialoprotein (BSP) for human embryonic stem cell culture. An RGD peptide sequence, that is arginylglycylaspartic acid, is a tripeptide composed of L-arginine, glycine, and L-aspartic acid.

In embodiments, the disclosure provides a stable synthetic microcarrier surface having a well defined composition and structure that can support serum-free adhesion and long term proliferation of human embryonic stem cells.

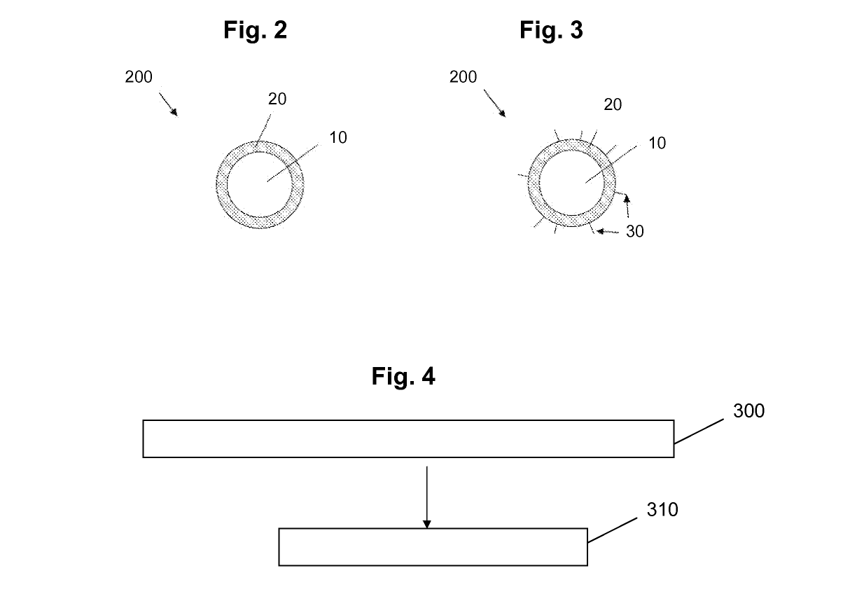

Referring to FIG. 2 and FIG. 3, a microcarrier 200 includes a base 10 and a coating 20 and can include a conjugated polypeptide 30. The coating 20 alone or coating 20 and polypeptide 30 together provide a surface to which cells can attach for the purposes of cell culture. In various embodiments, the coating layer 20 is deposited on or formed on a surface of an intermediate layer that is associated with the base material 10 via covalent or non-covalent interactions, either directly or via one or more additional intermediate layers (not shown). In such instances, the intermediate is considered, for the purposes of this disclosure, to be a part of the microcarrier base 10.

Microcarriers can have any suitable density. However, in a particularly useful application the microcarriers can have a density slightly greater than the cell culture medium in which they are to be suspended to facilitate separation of the microcarriers from the surrounding medium. In embodiments, the microcarriers can have a density of about 1.01 to about 1.10 grams per cubic centimeter. Microcarriers having such a density should be readily maintained in suspension in cell culture medium with gentle stirring.

In embodiments, it is particularly useful that the size variation of the microcarriers be relatively small to ensure that most, if not all, of the microcarriers can be suspended with gentle stirring. For example, the geometric size distribution of the microcarriers can be from about 1 to about 1.4. Microcarriers can be of any suitable size. For example, microcarriers can have a diametric dimension of between about 20 microns and 1,000 microns. Spherical microcarriers having such diameters can support the attachment of several hundred to thousands of cells per microcarrier. The size of the microcarrier bases, and thus the overall microcarrier, can be readily controlled via known techniques. For example, the physical characteristics of microcarrier bases formed via water-in-oil copolymerization techniques can be easily adjusted by varying the stirring speed or the type of emulsifier selected. For example, higher stirring speeds tend to result in smaller particle size. In addition, it is believed that the use of polymeric emulsifiers, such as ethylcellulose, enable larger particles relative to lower molecular weight emulsifiers. Accordingly, one can readily modify stirring speed or agitation intensity and emulsifier to obtain microcarrier bases of a desired particle size.

Microcarriers can be porous or non-porous. “Non-porous” refers to having substantially no pores or pores of an average size smaller than a cell with which the microcarrier is cultured, e.g., less than about 0.5 to about 1 micrometers. Non-porous microspheres are desired when the microcarriers are not degradable, because cells that enter pores of macroporous microcarriers can be difficult to remove. However, if the microcarriers are degradable, e.g. if they include an enzymatically or otherwise degradable cross-linker, it can be desirable for the microcarriers to be macroporous.

### 2. Surface Modified Microcarriers for Undifferentiated Human Embryonic Stem Cells.

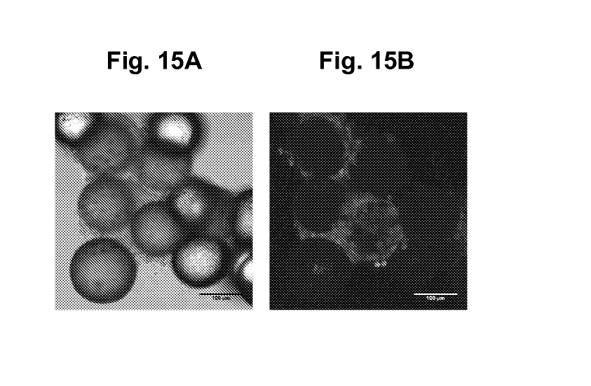

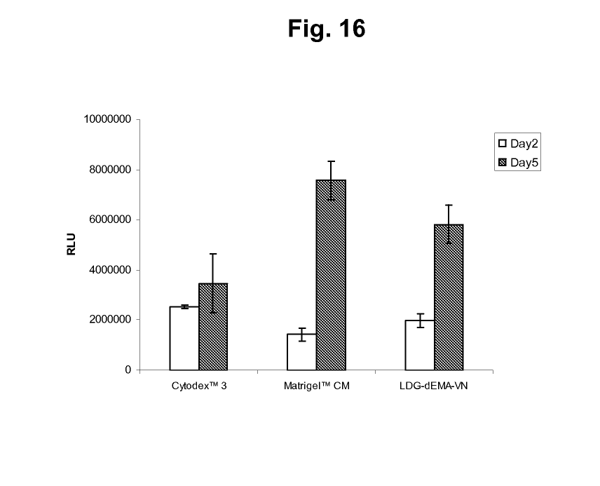

In embodiments, the disclosure provides a method for the preparation and use of synthetic peptide-derived microcarriers that can support serum-free culture of human embryonic stem cells (hESC). The microcarrier surfaces were prepared by direct conjugation of peptide sequences selected to mimic sequences of, for example, laminin derived from Vitronectin Ac-KGGPQVTRGDVTMP-NH₂ (VN), and bone sialoprotein Ac-KGGNGEPRGDTYRAY (BSP) to derivatized-maleic anhydride coated microcarriers. The sequence Ac-KGGPQVTRGDVTMP-NH₂ (VN) showed comparable adhesion, growth and expansion (FIG. 15) of human embryonic stem cells under serum-free conditions comparable to the freshly prepared Matrigel™ coated glass microcarrier control (FIG. 16), and superior to Cytodex™ 3 (collagen coated dextran beads) as confirmed by phase contrast, fluorescent, and confocal microscopy. This is significant as there are apparently no commercially available purely synthetic microcarriers for human embryonic stem cell expansion in chemically-defined media.

The peptide sequences Ac-KGGNGEPRGDTYRAY (BSP) and Ac-KGGPQVTRGDVTMP-NH₂ (VN), have been identified as excellent surface modifiers for human embryonic stem cells. When the VN peptide sequence was conjugated to a surface associated polymer, such as dEMA, they supported serum-free specific attachment and expansion of undifferentiated human embryonic stem cells. The results for each conjugated peptide sequence was comparable to Matrigel™ coated control beads (coated according to the manufacturer's standard protocol). Matrigel™ is a solubilized basement membrane preparation from BD Biosciences extracted from the mouse sarcoma, a tumor rich in extracellular matrix proteins which includes laminin (a major component), collagen IV, heparin sulfate proteoglycans, and entactin/nidogen.

Embryonic stem cells (ESCs), including human embryonic stem cells (hESCs), are able to grow and self-renew unlimitedly; they can be propagated in culture for extended periods and have an ability to differentiate to multiple cell types. However, these cells have specific cell culture needs. Slight changes in culture conditions can cause these cells to differentiate, or exhibit reduced growth and propagation characteristics. In many cases, ESC cultures require the addition of animal-derived materials either in or on a cell culture surface to effectively grow in culture. These animal-derived materials can harbor pathogens such as infective proteins and viruses, including retroviruses. Although some microcarriers have demonstrated the ability to facilitate proliferation of ESC in both un-differentiated (pluripotent) and differentiated states, they can still be considered inadequate for cell cultures that are directed toward the development of cell therapeutics in humans because of the threat of pathogens that might be carried from an animal source of cell culture additives to the cultured cells, to an individual treated with those cells. In addition, these animal-derived surfaces can have high lot-to-lot variability making results less reproducible, and they can be very expensive. In light of these disadvantages, surfaces that include animal-derived materials can be relegated to academic and pre-clinical research and can not be useful to produce, for example, stem cells to treat patients. Furthermore, because of the costs associated with these animal derived surfaces, they are considered very expensive even for academic research, providing opportunities for less costly and safer alternatives. Therefore, to provide a product which eliminates the risks associated with animal derived products, (meth)acrylate surfaces with special surface attributes, and improved methods of making these surfaces are provided.

In embodiments, cell culture surfaces can be made from ingredients which are not animal-derived, can sustain at least 15 passages of cells in cell culture, can be reliable and reproducible, and can allow for the growth of cells which show normal characteristics, normal karyotype, after defined passages. Cell culture surfaces for stem cells can be made from ingredients which are not animal-derived, and sustain undifferentiated growth of ES cells for at least 10 passages in culture. In embodiments, cell culture surfaces can also be stable. Cell culture surfaces can be non-toxic. They can withstand processing conditions including stem, radiation, or gas sterilization, possess adequate shelf life, and maintain quality and function after normal treatment. In addition, the cell culture surfaces can be suitable for large-scale industrial production. They can be scalable and cost effective to produce. The materials can also possess chemical compatibility with aqueous solutions and physiological conditions found in cell culture environments.

Cell culture studies conducted on synthetic surfaces have demonstrated that surface properties of microcarriers can affect the success of cell culture and can affect characteristics of cells grown in culture. For example, surface properties can elicit cell adhesion, spreading, growth, and differentiation of cells. Research conducted with human fibroblast cells 3T3 and HT-1080 fibrosarcoma cells has shown correlation with surface energetics, contact angle, surface charge, and modulus (Altankov, G., et al., The role of surface zeta potential and substratum chemistry for regulation of dermal fibroblasts interaction, Mat.-wiss. U. Werkstofftech., 2003, 34, 12, 1120-1128.) Anderson et al (US2005/0019747) disclosed depositing microspots of (meth)acrylates, including polyethylene glycol (meth)acrylates, onto a substrate as surfaces for stem cell-based assays and analysis. Self-Assembled Mono-layers (SAMS) surfaces with covalently linked laminin adhesive peptides have been used to enable adhesion and short-term growth of undifferentiated hES cells (Derda, S., et al., Defined Substrates for Human Embryonic Stem Cell Growth Identified from surface Arrays, ACS Chemical Biology, Vol. 2, No. 5, May 2, 2007, pp 347-355).

In embodiments, the disclosure provides anhydride polymer peptide-modified microcarriers that impart specific physical and chemical attributes to the surface, and methods of making these surfaces. These attributes can facilitate the proliferation of difficult-to-culture cells, such as undifferentiated hESCs. These anhydride polymer peptide-modified surfaces can be prepared with different properties. The anhydride polymer, peptide conjugate, and blocking agents have particular characteristics which, when combined and coated provide maleic anhydride peptide-modified surfaces that are amenable for cell culture. These characteristics can include, for example, hydrophilicity or hydrophobicity, positive charge, negative charge, or no charge (neutral), and compliant or rigid surfaces. For example, blocking agents or combinations of peptides which are hydrophilic can provide cell culture surfaces that are superior. Or, blocking agents or combinations of peptides which carry a charge can be superior. Or, blocking agents or combinations of peptides which influence swelling of the maleic anhydride polymer can influence the range of modulus or hardness can be superior. Or, monomers or blocking agents or combinations of peptides which exhibit a combination of these attributes can be superior.

### 3. Microcarrier Base

Any suitable microcarrier base can be used. In embodiments, the microcarrier base can be glass, ceramic, metal, polymeric, or combinations thereof. Examples of polymeric materials include, for example, polystyrenes, acrylates such as polymethylmethacrylate, acrylamides, agarose, dextrans, gelatins, latexes, and like materials, or combinations thereof. The microcarrier base can have special characteristics, such as being magnetic, to facilitate separation from bulk media. In embodiments, the microcarriers can be microspheres, many of which are commercially available. Microspheres can be produced by any suitable method such as suspension polymerization of an “water-in-oil” emulsion.

Many suitable functionalized microcarrier substrates are available from commercial sources. For example, COOH, SH, NH₂, and CHO functionalized polystyrene resins and microspheres are available from Rapp Polymere GMBH; amino, carboxylate, carboxy-sulfate, hydroxylate, and sulfate functionalized polystyrene beads are available from Polysciences, Inc.; and amine functionalized glass beads are available from Polysciences, Inc. Carboxylate functionalized dextran beads are available from GE Healthcare, Hyclone, and Sigma-Aldrich. Azlactone functionalized beads are available from Pierce. Unfunctionalized magnetic beads are available from Merck.

If desired, additional functional groups can readily be added to microcarriers via known techniques. For example, glass carriers can be readily functionalized with an appropriate organosilane. It can be desirable to treat or etch the surface of the glass carrier prior to functionalization to increase surface area. Functionalized epoxy resins can be employed to functionalize glass or other suitable microcarriers. Polystyrene or other suitable microcarriers can also be readily functionalized using known techniques. For example, a microcarrier base can be prepared by polymerization of monomers such as chloromethylstyrene or 4-t-BOC-hydroxystyrene. Other suitable monomers include styrene, a-methylstyrene, or other substituted styrene or vinyl aromatic monomers that, after polymerization, can be chloromethylated to produce a reactive microcarrier intermediate that can subsequently be converted to a functionalized microcarrier. If desired, monomers not bearing reactive groups (including the crosslinking agent) can be incorporated into the microcarrier. Chemical modification of the reactive microcarrier intermediate can be carried out by a variety of conventional methods.

For optical or electrical detection applications, the microcarrier can be transparent, impermeable, or reflecting, as well as electrically conducting, semiconducting, or insulating. For biological applications, the microcarrier material can be either porous or nonporous and can be selected from either organic or inorganic materials.

In embodiments, the microcarrier can be composed of an inorganic material. Examples of inorganic microcarrier materials include, but are not limited to, metals, semiconductor materials, glass, and ceramic materials. Examples of metals that can be used as microcarrier materials include, but are not limited to, gold, platinum, nickel, palladium, aluminum, chromium, steel, and gallium arsenide. Semiconductor materials used for the microcarrier material can include, for example, silicon and germanium. Glass and ceramic materials used for the microcarrier material can include, for example, quartz, glass, porcelain, alkaline earth aluminoborosilicate glass and other mixed oxides. Further examples of inorganic microcarrier materials include graphite, zinc selenide, mica, silica, lithium niobate, and inorganic single crystal materials. In embodiments, the microcarrier can be made of gold such as, for example, a gold sensor chip.

For hydroxyl containing inorganic microcarriers, factors such as initial concentration of surface hydroxyls, type of surface hydroxyls, stability of the bond formed, and dimensions or features of the microcarrier can influence the effectiveness of the tie layer or polymer coating. It can be desirable to have the maximum number of accessible reactive sites on the glass microcarrier to maximize initiator coupling. Acid or base etching (e.g., 1M sodium hydroxide, ammonia, hydrochloric acid), UV-ozone, or plasma treatment can be included as a glass microcarrier pretreatment step to clean the surface, expose more reactive silanol groups, or both, which can influence the subsequent interaction of the surface with a tie-layer or polymer coating. Other hydroxyl-containing microcarriers such as silica, quartz, aluminum, alumino-silicates, copper inorganic oxides, and like materials, or combinations thereof, can be used as an alternative to glass.

In embodiments, the microcarrier can be a porous, inorganic layer. Any of the porous substrates and methods of making such substrates disclosed in commonly owned U.S. Pat. No. 6,750,023, can be used herein. In embodiments, the inorganic layer on the microcarrier can be a glass or metal oxide. In embodiments, the inorganic layer can be a silicate, an aluminosilicate, a boroaluminosilicate, a borosilicate glass, or a combination thereof. In embodiments, the inorganic layer can be TiO₂, SiO₂, Al₂O₃, Cr₂O₃, CuO, ZnO, Ta₂O₅, Nb₂O₅, ZnO₂, or a combination thereof. In embodiments, the microcarrier can be SiO₂ with a layer comprising Ta₂O₅, Nb₂O₅, TiO₂, Al₂O₃, silicon nitride, or a mixture thereof, wherein the layer is adjacent to the surface of the SiO₂. The silicon nitride can be represented by the formula SiN_{X}, where the stoichiometry of silicon and nitrogen can vary.

In embodiments, the microcarrier can be composed of an organic material. Useful organic materials can be made from polymeric materials due to their dimensional stability and resistance to solvents. Examples of organic microcarrier materials include, for example, polyesters, such as polyethylene terephthalate and polybutylene terephthalate; polyvinylchloride; polyvinylidene fluoride; polytetrafluoroethylene; polycarbonate; polyamide; poly(meth)acrylate; polystyrene, polyethylene; or ethylene/vinyl acetate copolymer.

### 4. Binding Polymer

In embodiments, a binding polymer comprising one or more reactive groups that can bind a peptide to the microcarrier can be directly or indirectly attached to the microcarrier. The “reactive group” on the binding polymer permits the attachment of the binding polymer to the peptide. The reactive groups can also facilitate the attachment of the binding polymer to the microcarrier. In embodiments, the binding polymer can be attached covalently, electrostatically, or both, to the microcarrier. The binding polymer can have one or more different reactive groups. In embodiments, two or more different binding polymers can be attached to the micro carrier.

In embodiments, the binding polymer reactive group(s) can form a covalent bond with a nucleophile such as, for example, an amine or thiol. The amine or thiol can be derived from the biomolecule or a molecule that is attached to the surface of the microcarrier (e.g., a tie-layer) and used to indirectly attach the binding polymer to the microcarrier. Examples of reactive groups include, for example, an anhydride group, an epoxy group, an aldehyde group, an activated ester (e.g., n-hydroxysuccinimide (NHS), an isocyanate, an isothiocyanate, a sulfonyl chloride, a carbonate, an aryl halide, alkyl halide, an aziridine, a maleimide, and like groups, or combinations thereof. In embodiments, two or more different chemical types of reactive groups can be present on the binding polymer.

A particularly useful binding polymer is a synthetic coating free from animal-derived components. Animal derived components can contain viruses or other infectious agents or can provide a high level of batch-to-batch variability. In embodiments, the coating is a maleic anhydride based coating, e.g., as described in U.S. Pat. No. 7,781,203.

Also present on the binding polymer is a plurality of ionizable or ionic groups. Ionizable or ionic groups are groups that can be converted to a charged (i.e., ionic) group under particular reaction conditions. For example, a carboxylic acid (an ionizable group) can be converted to the corresponding carboxylate (charged or ionic group) by treating the acid with a base. The charged groups can be either positive or negative. An example of a positively charged group is an ammonium group. Examples of negatively charged groups include carboxylate, sulfonate, and phosphonate groups. In embodiments, two or more different ionizable or ionic groups can be present on the binding polymer.

The binding polymer can be water-soluble, water-insoluble, or amphiphile, depending, for example, upon the technique used to attach the binding polymer to the microcarrier. The binding polymer can be either linear or non-linear. For example, when the binding polymer is non-linear, the binding polymer can be branched, hyperbranched, crosslinked, dendritic polymer, or combination thereof. The binding polymer can be a homopolymer or a copolymer of two or more suitable monomer types.

In embodiments, the binding polymer can be a copolymer comprised of maleic anhydride and a first monomer. In embodiments, the amount of maleic anhydride in the binding polymer can be, for example, from 5% to 50%, 5% to 45%, 5% to 40%, 5% to 35%, 5% to 30%, 5% to 25%, 10% to 50%, 15% to 50%, 20% to 50%, 25% to 50%, or 30% to 50% by stoichiometry (i.e., relative mole equivalents) of the first monomer. In embodiments, the first monomer selected improves the stability of the maleic anhydride group in the binding polymer. In embodiments, the first monomer can reduce nonspecific binding of the biomolecule to the microcarrier. In embodiments, the amount of maleic anhydride in the binding polymer can about 50% of the first monomer. In embodiments, the first monomer can be, for example, styrene, tetradecene, octadecene, methyl vinyl ether, triethylene glycol methyl vinyl ether, butylvinyl ether, divinylbenzene, ethylene, dimethylacrylamide, vinyl pyrrolidone, a polymerizable oligo(ethylene glycol) or oligo(ethylene oxide), propylene, isobutylene, vinyl acetate, methacrylate, acrylate, acrylamide, methacrylamide, or a combination thereof.

In embodiments, the binding polymer can be, for example, poly(vinyl acetate-maleic anhydride), poly(styrene-co-maleic anhydride), poly(isobutylene-alt-maleic anhydride), poly(maleic anhydride-alt-1-octadecene), poly(maleic anhydride-alt-1-tetradecene), poly(maleic anhydride-alt-methyl vinyl ether), poly(triethyleneglycol methylvinyl ether-co-maleic anhydride), poly(ethylene-alt-maleic anhydride), or a combination thereof.

5. Microcarrier with Binding Polymer Coating

A polymer layer can be disposed on a surface of a microcarrier base via any process. In embodiments, the coating provides a uniform layer that does not delaminate during typical cell culture conditions. The coating layer can be associated with the microcarrier base via covalent or non-covalent interactions. Examples of non-covalent interactions that can associate the binding polymer with the microcarrier include, for example, chemical adsorption, hydrogen bonding, surface interpenetration, ionic bonding, van der Waals forces, hydrophobic interactions, dipole-dipole interactions, mechanical interlocking, and combinations thereof. An example of a covalent interaction is a reaction of amino groups on the surface of the microcarrier with the anhydride group of the binding polymer to form an amide bond. An example of a dipole-dipole interaction is attraction of the negative charge of a ring opened anhydride group (i.e., carboxyl group —CO₂⁻) with a positive charged amine group (i.e., —NH⁺—) at the surface of the microcarrier.

In addition to the binding polymer that forms the coating layer, a composition forming the layer can include one or more additional compounds such as surfactants, wetting agents, polymerization initiators, catalysts or activators.

Binding polymers can be brought into contact with the functionalized base microcarrier. In embodiments, the base can be referred to as the “microcarrier” on which the tie-layer or binding polymer is deposited or formed. For example, the microcarrier can be suspended in the binding polymer solution and the microcarrier can be coated with the binding polymer through covalent reaction. As the binding polymer can be in the form of a solid or viscous liquid, it can be desirable to dilute the binding polymer in a suitable solvent to reduce viscosity prior to suspending the polymer with the base microcarrier. Reducing viscosity can allow for thinner and more uniform layers of the coating material to be formed. The solvent in particularly useful embodiments is compatible with the base material and the binding polymer. It can be desirable to select a solvent that is nontoxic to the cells to be cultured and that does not interfere with the coating reaction. Alternatively or additionally, selection of a solvent that can be substantially completely removed or removed to an extent that it is non-toxic or no longer interferes with coating can be desirable. It can also be desirable that the solvent be readily removed without harsh conditions, such as vacuum or extreme heat. Volatile solvents are examples of such readily removable solvents.

Some solvents that can be suitable in various situations for coating articles include, for example, 2-methyl N-pyrrolidinone (NMP), dimethylformamide (DMF), dimethylsulfoxide (DMSO), 2-propanol (IPA), methanol, ethanol, acetone, butanone, acetonitrile, 2-butanol, acetyl acetate, ethyl acetate, water or combinations thereof. The binding polymer is preferably inert to the solvent and the solvent does not hydrolyze the binding polymer or react with the binding polymer.

The binding polymer can be diluted with solvent by any suitable amount to achieve the desired viscosity, binding polymer concentration, or colloidal suspension. For example, the binding polymer solution can contain between about 0.1% to about 99% binding polymer. By way of example, the binding polymer can be diluted with an ethanol or other solvent to provide a composition having between about 0.1% and about 50% monomer, or from about 0.1% to about 10% binding polymer by volume, or from about 0.1% to about 1% binding polymer by volume. The monomers can be diluted with solvent so that the binding polymer coating layer achieves a desired thickness.

The binding polymer can be coated as a colloidal solution. A colloidal solution can be created by, for example, first dissolving the binding polymer into highly compatible solvent allowing it to fully dissolve, followed by dilution with a poor solvent that partially precipitates the polymer from the solution.

The microcarrier bound binding polymer coating can be washed with solvent one or more times to remove impurities such as unbound polymer or low molecular weight polymer species. In embodiments, the layer can be washed with ethanol, or ethanol water mixtures, such as greater than 90%, 95%, or 99% ethanol to water by volume. In embodiments, the washing solvent does not contain any water or nucleophilic species that can hydrolyze the unreacted reactive groups within the binding polymer. Hydrolysis can render the resulting surface unreactive towards a desired peptide. The size and shape of the base microcarrier can determine the washing method. For example, a flat sheet can be washed by dipping in solvent or washed by squirt bottle, spraying, or any other washing method. Any suitable filter apparatus can be used to remove the washing solvent from microparticle microcarriers. Examples of filter systems include peptide synthesis vessels equipped with a vacuum filter or a Soxhlet apparatus for higher temperature washings.

Referring to FIG. 4, the binding polymer can be coated (e.g., covalently bound) to the microcarrier base. In embodiments, a method for coating a polymer layer to a microcarrier includes applying a binding polymer coating to the microcarrier base (300). The method can further include conjugating a polypeptide to the polymer layer (310).

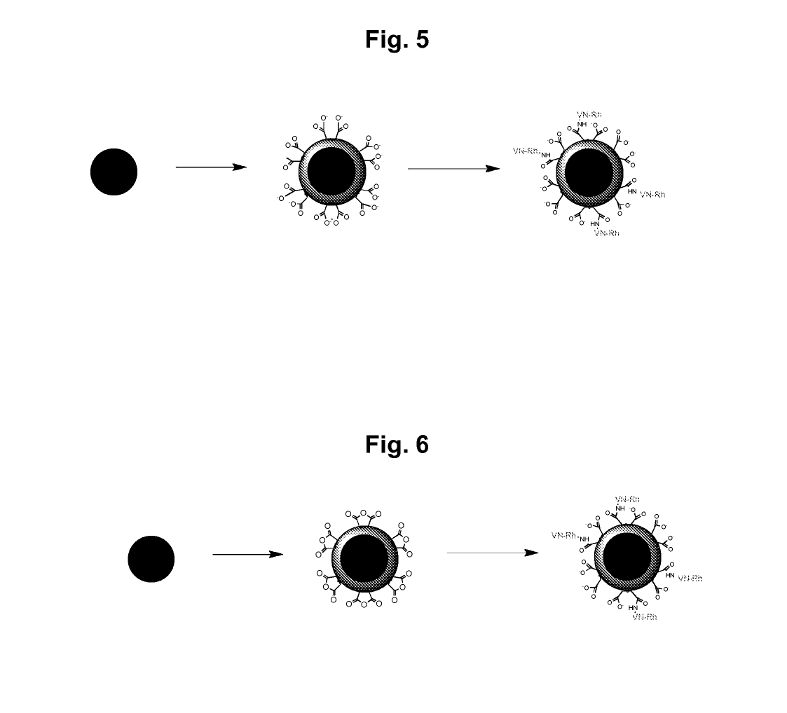

FIG. 5 shows an example of one suitable reaction scheme for coating an anhydride binding polymer on a microcarrier. An amino siloxane oligomer (APS), having silyl ether functionality can conjugated to a glass microcarrier (glass bead), leaving the bead with an amino functionality conjugated via an ether linkage. The amino-silane functionalized glass microcarrier can then placed in solution with an anhydride binding polymer (for example, dEMA) in an appropriate solvent (e.g., a mixture of 2-proponal/N′-methylpyrrolidinone) followed by water hydrolysis to produce a coated microcarrier. In this instance, the microcarrier coating has free carboxylic acid groups resulting from hydrolyzed anhydride, which groups provide for ready conjugation of polypeptides via activation chemistry (e.g., EDC/NHS).

FIG. 6. illustrates another example of a suitable reaction scheme for coating an anhydride binding polymer on a microcarrier. An amino siloxane oligomer (APS), having silyl ether functionality can be conjugated to a glass microcarrier (glass bead), leaving the bead with an amino functionality conjugated via an ether linkage. The amino-silane functionalized glass microcarrier can then be placed in suspension with a solution of an anhydride binding polymer (e.g., dEMA) in an appropriate solvent (e.g., a mixture of 2-proponal/N′-methylpyrrolidinone). In the absence of a hydrolyzing agent (e.g., water), an anhydride reactive coated microcarrier results. In this instance, this conjugation of polypeptides can take place without activation chemistry.

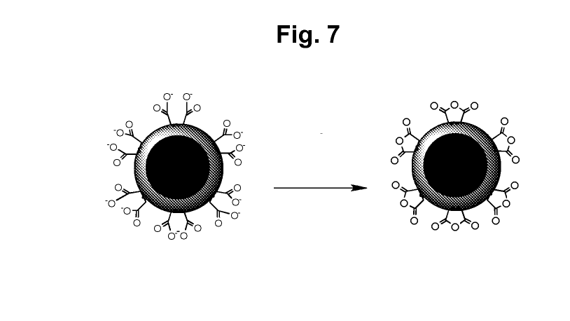

FIG. 7 illustrates that there can be instances where the anhydride surface can be completely hydrolyzed (left). The surface can be dehydrated to regenerate the anhydride groups (right). A hydrolyzed dEMA microcarrier bead was dried in a vacuum oven at 120° C. for 24 hours to regenerate the anhydride functionality prior to peptide conjugation. The high temperature and vacuum caused adjacent carboxylate groups to condense to an anhydride group. This can be important during the manufacturing of these types of surfaces where a long lag time can exist between the dEMA coating and peptide conjugation.

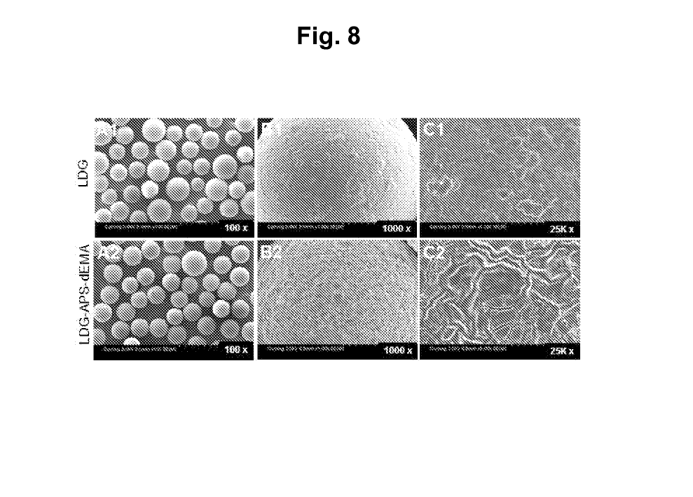

FIG. 8 shows scanning electron micrograph (SEM) images of amine functionalized low density glass microcarriers coated with dEMA. SEM was used to visualize the surface texture of the dEMA microcarriers before and after coatings. After the dEMA coating (FIG. 8, B2, C2, and D2) a clear difference in surface texture at 1,000× and 25,000× magnification was observed compared to the neat glass beads (FIG. 8, A1, B1, and C1). Specifically, distinct wrinkles were observed on the dEMA coated surfaces. Since the SEM images were captured on dry beads, the wrinkled surface can be due to the collapsing of the polymer.

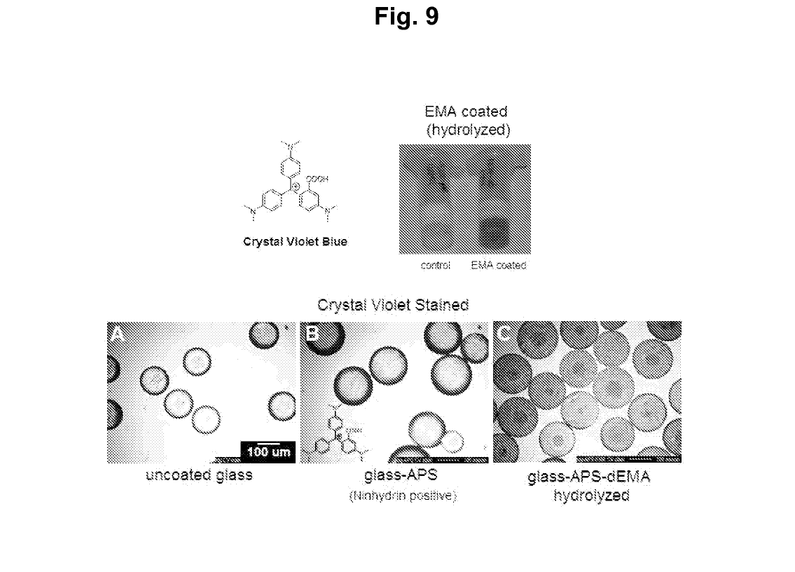

Crystal violet staining can be used to monitor various steps during anhydride polymer coating. Crystal violet is a positively charged, visible dye that binds ionically to negatively charged groups such as carboxylate group of hydrolyzed maleic anhydride polymers. Referring to FIG. 9, images of various crystal violet stained polystyrene (top) and—glass microcarriers (bottom) are presented. The chemical structure of crystal violet blue is also shown. The amine functionalized polystyrene microcarriers in bulk (Top—“control”) did not stain with crystal violet blue, while the EMA coated polystyrene microcarriers in bulk (Top—“EMA coated”) showed staining of the microcarriers. The Brightfield images on the bottom of FIG. 9 indicated no crystal violet staining on uncoated low density glass beads (A), and no crystal violet staining glass on beads coated with the aminosilane tie layer (B, amine functionalized). However, uniform crystal violet staining was observed on dEMA coated beads (C). Furthermore, both ldg-APS and ldg-APS-dEMA coated beads tested positive for free amines by ninhydrin.

The amount of binding polymer attached to the microcarrier can vary depending upon, for example, the selection of the binding polymer, the peptide, and the cell to be attached. In embodiments, the binding polymer can be, for example, at least one monolayer. In embodiments, the binding polymer can have a thickness of, for example, about 10 Å to about 2,000 Å. In embodiments, the thickness of the binding polymer can have a lower endpoint of, for example, 10 Å, 20 Å, 40 Å, 60 Å, 80 Å, 100 Å, 150 Å, 200 Å, 300 Å, 400 Å, or 500 Å, and an upper endpoint of, for example, 750 Å, 1,000 Å, 1,250 Å, 1,500 Å, 1,750 Å, or 2,000 Å, where any lower endpoint can be combined with any upper endpoint to form the thickness range, including intermediate values and ranges.

The binding polymer can be attached to the microcarrier using known techniques. For example, the microcarrier can be dipped in a solution of the binding polymer. In embodiments, the binding polymer can be sprayed, vapor deposited, screen printed, or robotically pin printed or stamped on the microcarrier. This could be done either on a fully assembled microcarrier or on a bottom insert (e.g., prior to attachment of the bottom insert to a holey plate to form a microplate).

In embodiments, the microcarrier support can be made by attaching a binding polymer directly or indirectly to the microcarrier, wherein the binding polymer has a plurality of reactive groups capable of attaching to a biomolecule. When the binding polymer is directly or indirectly attached to the microcarrier, the binding polymer can be attached either covalently or non-covalently (e.g., electrostatically). FIG. 1 shows an aspect of the attachment of the binding polymer to the microcarrier, where a nucleophilic group (Sur) (e.g., an amino group, hydroxyl group, or thiol group) reacts with an anhydride group of the binding polymer to produce a new covalent bond.

In embodiments, when the binding polymer is indirectly attached to the microcarrier, a tie layer can be used. The tie layer can be covalently or electrostatically attached to the outer surface of the microcarrier. The term “outer surface” with respect to the microcarrier is the region of the microcarrier that is exposed and can be subjected to manipulation. For example, any surface on the microcarrier that can come into contact with a solvent or reagent upon contact is considered the outer surface of the microcarrier. Thus, the tie layer can be attached to the microcarrier and the binding polymer.

In embodiments, the disclosed microcarriers can have a tie layer covalently bonded to the microcarrier. However, it is also contemplated that a different tie layer can be attached to the microcarrier by other means in combination with a tie layer that is covalently bonded to the microcarrier. For example, one tie layer can be covalently bonded to the microcarrier and a second tie layer can be electrostatically bonded to the microcarrier. In embodiments, when the tie layer is electrostatically bonded to the microcarrier, the compound used to make the tie layer can be positively charged and the outer surface of the microcarrier can be treated such that a net negative charge exists so that tie layer compound and the outer surface of the microcarrier form an electrostatic bond.

In embodiments, the outer surface of the microcarrier can be chemically modified (i.e., derivatized) so that there are groups capable of forming a covalent bond with the tie layer. The tie layer can be directly or indirectly covalently bonded to the micro carrier. In situation where the tie layer is indirectly bonded to the micro carrier, a linker possessing groups that can covalently attach to both the microcarrier and the tie layer can be used. Examples of linkers include, for example, an ether group, a polyether group, a polyamine, or a polythioether. If a linker is not used, and the tie layer can be covalently bonded to the microcarrier, and is referred to as direct covalent attachment.

In embodiments, the tie layer can be derived from a compound comprising one or more reactive functional groups that can react with the binding polymer. The phrase “derived from” with respect to the tie layer means the resulting residue or fragment of the tie layer compound when it is attached to the microcarrier. The functional groups permit the attachment of the binding polymer to the tie layer. In embodiments, the functional groups of the tie layer compound can be, for example, an amino group, a thiol group, a hydroxyl group, a carboxyl group, an acrylic acid, an organic and inorganic acid, an activated ester, an anhydride, an aldehyde, an epoxide, an isocyanate, an isothiocyanate, or salts thereof, and like groups, or a combination thereof.

In embodiments, the microcarrier can be amine-modified with, for example, a polymer comprising at least one amino group. Examples of such polymers include, for example, poly-lysine, polyethyleneimine, poly(allyl)amine, or silylated polyethyleneimine. In embodiments, the microcarrier can be modified with an aminosilane. In embodiments, the tie layer can be derived from a straight or branched-chain aminosilane, aminoalkoxysilane, aminoalkylsilane, aminoarylsilane, aminoaryloxysilane, or a derivative or salt thereof. In embodiments, the tie layer can be derived from 3-aminopropyl trimethoxysilane, N-(beta-aminoethyl)-3-aminopropyl trimethoxysilane, N-(beta-aminoethyl)-3-aminopropyl triethoxysilane, N′-(beta-aminoethyl)-3-aminopropyl methoxysilane, or aminopropylsilsesquixoxane, or like compounds.

In embodiments, when the microcarrier is comprised of gold, the binding polymer can be attached to the microcarrier by an aminothiol such as, for example, 11-amino-1-undecanethiol hydrochloride.

The tie layer can be attached to any of the disclosed microcarriers using known techniques. For example, the microcarrier can be dipped in a solution of the tie layer compound. In a further aspect, the tie layer compound can be sprayed, vapor deposited, screen printed, or robotically pin printed or stamped on the microcarrier. This can be done either on a fully assembled microcarrier or on a bottom insert (e.g., prior to attachment of the bottom insert to a holey plate to form a microplate).

In embodiments, the microcarrier can be a gold chip, the binding polymer can be poly(ethylene-alt-maleic anhydride) indirectly attached to the microcarrier by an aminothiol, and the ratio of reactive groups to ionizable groups in the binding polymer can be, for example, from 0.67 to 3.0. In embodiments, the microcarrier can be a glass microcarrier with a layer comprising Ta₂O₅, Nb₂O₅, TiO₂, Al₂O₃, silicon nitride, SiO₂ or a mixture thereof, the binding polymer can be poly(ethylene-alt-maleic anhydride) indirectly attached to the microcarrier by a tie layer, where the tie layer is derived from aminopropylsilane (e.g., gamma-aminopropylsilane, GAPS), and the ratio of reactive groups to ionizable groups in the binding polymer can be from 0.67 to 3.0. In embodiments, the poly(ethylene-alt-maleic anhydride) can be preblocked with methoxyethyl amine, or like pre-block agents, prior to attaching the polymer to the microcarrier.

The binding polymers useful herein do not contain a photoreactive group. Photoreactive groups respond to specific applied external stimuli to undergo active species generation with resultant covalent bonding to an adjacent chemical structure, e.g., as provided by the same or a different molecule. Photoreactive groups are those groups of atoms in a molecule that retain their covalent bonds unchanged under conditions of storage; however, upon activation by an external energy source, form covalent bonds with other molecules. The photoreactive groups generate active species such as free radicals and particularly nitrenes, carbenes, and excited states of ketones upon absorption of electromagnetic energy.

If desired, the microcarrier bulk density can be controlled by the amount of binding polymer bound to the surface. For example, the level of binding polymer bound to the microcarrier can be controlled by the time the base microcarrier is exposed to the binding polymer solution. Alternatively, the concentration of the binding polymer solution can affect the amount of binding polymer that is bound to the microcarrier at a given time. The microcarriers can have a density of about 0.95 to 1.10 grams per cubic centimeter.

### 6. Ratio of Reactive Groups to Ionizable (Ionic) Groups

In embodiments, the relative mole ratio of reactive groups to ionizable groups can be, for example, from 0.5 to 5.0. In embodiments, a lower mole ratio can be 0.5, 0.6, 0.7, 0.8, 0.9, 1.0, 1.5, 2.0, 2.5, 3.0, 3.5, 4.0, 4.5, or 5.0, and the upper ratio can be 2.0, 2.5, 3.0, 3.5, 4.0, 4.5, 5.0, 5.5, 6.0, 6.5, 7.0, 7.5, 8.0, 8.5, 9.0, 9.5, or 10.0, where any lower and upper ratio can form a ratio range, including intermediate values and ranges. In embodiments, the mole ratio range of reactive groups to ionizable groups can be from 0.5 to 9.0, 0.5 to 8.0, 0.5 to 7.0, 0.5 to 6.0, 0.5 to 5.0, 0.5 to 4.0, 0.5 to 3.0, 0.6 to 3.0, 0.65 to 3.0, or 0.67 to 3.0.

The formation and number of reactive groups and ionizable groups present on the binding polymer can be controlled in a number of ways. In embodiments, the binding polymer can be prepared from monomers having reactive groups and monomers with ionizable groups. In embodiments, the stoichiometry of the monomers selected can control the ratio of reactive groups and ionizable groups. In embodiments, a polymer having just reactive groups can be treated, for example, pre-blocked, so that some of the reactive groups are converted to ionizable groups prior to attaching the binding polymer to the microcarrier. The starting polymer can be commercially available or synthesized using known techniques. In embodiments, a polymer can be attached to the microcarrier, and the attached polymer can be treated with various reagents to add either reactive groups and ionizable groups or convert reactive groups to ionizable groups. In embodiments, the binding polymer that possesses reactive groups can be attached to the micro carrier, where the microcarrier reacts with the reactive groups and produces ionizable groups.

For example as shown in FIG. 1, when a polymer with a repeat unit of R′-maleic anhydride, where R′ can be a residue of an unsaturated monomer selected among monomers able to copolymerize with maleic anhydride such as, for example, ethylene, propylene, isobutylene, octadecene, tetradecene, vinyl acetate, styrene, vinyl ethers such as methyl vinyl ether, butyl vinyl ether, triethylene glycol vinyl ether, (meth)acrylates, (meth)acrylamide, vinyl pyrrolidinone, polymerizable oligo(ethylene glycol) or oligo(ethylene oxide) is reacted with W—R, where W is a nucleophilic group such as, for example, NH₂, OH, or SH and R can be hydrogen or a substituted or unsubstituted alkyl group (linear or branched) having, for example, 6 carbon atoms, an oligo(ethylene oxide) or oligo(ethylene glycol), or a dialkyl amine such as dimethyl amino propyl or diethyl amino propyl, the anhydride ring-opens and produces the carboxylic acid (an ionizable group). This step is referred to as pre-blocking. The pre-blocked polymer can then be applied to the surface of the microcarrier. Referring to FIG. 1, if the microcarrier surface has nucleophilic groups (Sur), where Sur can be for example NH₂, OH, or SH, these groups can react with the maleic anhydride groups present on the pre-blocked polymer to form a covalent bond between the pre-blocked polymer and the microcarrier.

The ratio of reactive groups to ionizable groups can be controlled by using specific amounts of reagents. Other properties of the binding polymer (e.g., hydrophobicity) can be altered as needed by controlling the starting materials used to prepare the binding polymer (e.g., selection of hydrophobic monomers) or by appropriate choice of the derivatizing or blocking reagent. In embodiments, the ratio of reactive groups to ionizable groups can be controlled by converting the one or more reactive groups to inactive groups. In embodiments, from about 10% to about 50% of the reactive groups present on the binding polymer can be blocked or rendered inactive. The term “blocked” refers to the conversion of a reactive group present on the binding polymer to an inactive group, where the inactive group does not form a covalent attachment with a biomolecule. In various aspects, the amount of reactive groups that can be blocked can be, for example, 10%, 12%, 14%, 16%, 18%, 20%, 22%, 24%, 26%, 28%, 30%, 32%, 34%, 36%, 38%, 40%, 42%, 44%, 46%, 48%, or about 50%, including intermediate values and ranges, where any value can form a lower and upper end of the relative mole ratio of a range. In embodiments, from about 10% to about 45%, about 10% to about 40%, about 10% to about 35%, about 15% to about 35%, about 20% to about 35%, or about 25% to about 35%, including intermediate values and ranges, of the reactive groups are blocked.

In embodiments, the blocking agent can react with the binding polymer prior to attaching the binding polymer to the microcarrier, or alternatively, the binding polymer can be attached to the microcarrier first followed by blocking with the blocking agent. In embodiments, the blocking agent can be at least one nucleophilic group, the binding polymer comprises at least one electrophilic group, and the blocking agent is attached to the binding polymer by a reaction between the electrophilic group and the nucleophilic group. In embodiments, the blocking agent can be covalently attached to the binding polymer. For example, when the blocking agent comprises an amine group, hydroxyl group, or thiol group, it can react with an electrophilic group present on the binding polymer (e.g., an epoxy, anhydride, activated ester group) to produce a covalent bond.

In embodiments, the blocking agent can be an alkyl amine, an alkylhydroxy amine, or an alkoxyalkyl amine. The term “alkyl” as used herein refers to a branched or unbranched saturated hydrocarbon group having form 1 to 25 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl, and like groups. Examples of longer chain alkyl groups include, for example, an oleate group or a palmitate group. A “lower alkyl” group is an alkyl group containing from one to six carbon atoms. The term “alkylhydroxy” refers to an alkyl group where at least one of the hydrogen atoms is substituted with a hydroxyl group. The term “alkylalkoxy” as used herein is an alkyl group as defined above where at least one of the hydrogen atoms is substituted with an alkoxy group —OR, where R is an alkyl group.

In embodiments, the blocking agent can be, for example, ammonia, 2-(2-aminoethoxy)ethanol, N,N-dimethyl ethylenediamine, ethanolamine, ethylenediamine, hydroxyl amine, methoxyethyl amine, ethyl amine, isopropyl amine, butyl amine, propyl amine, hexyl amine, 2-amino-2-methyl-1-propanol, 2-(2-aminoethyl amino)ethanol, 2-(2-aminoethoxy)ethanol, dimethylethanolamine, dibutyl ethanolamine, 1-amino-2-propanol, polyethylene glycol, polypropylene glycol, 4,7,10-trioxa-1,13-tridecanediamine, polyethylene glycol or an amine-terminated-polyethylene glycol, Trizma hydrochloride, or combinations thereof. In embodiments, the blocking agent can be, for example, water, H₂S, an alcohol (ROH), or alkyl thiol (RSH), where R is an alkyl group.

The disclosed microcarrier supports having a ratio of reactive groups to ionizable groups present on the binding polymer can have numerous advantages over known microcarrier supports. The ratio of reactive groups to ionizable groups permits increased loading or attachment (directly or indirectly with the use of a tie layer) of the binding polymer to the microcarrier. The attachment of the binding polymer to the. microcarrier involves mild conditions and does not require preactivation with, for example, EDC/NHS. This can save time and reduce costs with respect to manufacturing the supports. It is also possible to control the relative mole ratio of reactive groups to ionizable groups with other properties of the binding polymer such as hydrophobicity/hydrophilicity, which can increase the cell culture efficiency of the support.

In embodiments, the disclosed microcarrier supports can have a higher binding capacity between the support and a cell. It is believed that if more binding polymer can be loaded on the microcarrier then more cells can be attached to the binding polymer and the microcarrier. If more cells can be attached to the microcarrier, the performance of the support can also be enhanced. In embodiments, once the cell is attached to or associated with the binding polymer, the immobilized cell can more easily handled or manipulated.

7. Peptide Conjugation with Coated Microcarrier

Any suitable polypeptide can be conjugated to a coated microcarrier. In embodiments, polypeptides or proteins can be synthesized or obtained through recombinant techniques to provide synthetic, non-animal-derived materials. Particularly useful polypeptides include an amino acid capable of conjugating to the coating; e.g., via the free carboxyl group formed from the hydrolyzed anhydride group of the coating. For example, any native or biomimetic amino acid having functionality that enables nucleophilic addition, e.g. via amide bond formation, can be included in polypeptide for purposes of conjugating to the coating. Lysine, homolysine, ornithine, diaminoproprionic acid, and diaminobutanoic acid, are examples of amino acids having suitable properties for conjugation to a carboxyl group of the microcarrier. In addition, the N-terminal alpha amine of a polypeptide can be used to conjugate to the carboxyl group, if the N-terminal amine is not capped. In embodiments, the amino acid of polypeptide that conjugates with the coating is at the carboxy terminal position or the amino terminal position of the polypeptide.

In embodiments, the polypeptide, or a portion thereof, has cell adhesive activity, i.e., when the polypeptide is conjugated to the coated micro carrier, the polypeptide allows a cell to adhere to the surface of the peptide-containing coated microcarrier. For example, the polypeptide can include an amino sequence, or a cell adhesive portion thereof, recognized by proteins from the integrin family or leading to an interaction with cellular molecules able to sustain cell adhesion. For example, the polypeptide can include, for example, an amino acid sequence derived from collagen, keratin, gelatin, fibronectin, vitronectin, laminin, bone sialoprotein (BSP), or the like, or portions thereof. In embodiments, polypeptide includes an amino acid sequence of Arg-Gly-Asp (RGD).

In embodiments, the disclosed microcarriers provide a synthetic surface to which any suitable adhesion polypeptide or combinations of polypeptides can be conjugated, providing an alternative to biological substrates or serum that have unknown components. In current cell culture practice, it is known that some cell types require the presence of a biological polypeptide or combination of peptides on the culture surface for the cells to adhere to the surface and be sustainably cultured. For example, HepG2/C3A hepatocyte cells can attach to plastic culture ware in the presence of serum. It is also known that serum can provide polypeptides that can adhere to plastic culture ware to provide a surface to which certain cells can attach. However, biologically-derived substrates and serum contain unknown components. For cells where the particular component or combination of components (peptides) of serum or biologically-derived substrates that cause cell attachment are known, those known polypeptides can be synthesized and applied to a disclosed microcarrier to allow the cells to be cultured on a synthetic microcarrier having no or very few components of unknown origin or composition.

A polypeptide can be conjugated to the coated microcarrier via any suitable technique. A polypeptide can be conjugated to a polymerized microcarrier by, for example, an amino terminal amino acid, a carboxy terminal amino acid, or an internal amino acid. One suitable technique involves 1-ethyl-3-[3-dimethylaminopropyl]carbodiimide hydrochloride (EDC)/N-hydroxysuccinimide (NHS) chemistry, as generally known in the art. EDC and NHS or N-hydroxysulfosuccinimide (sulfo-NHS) can react with carboxyl groups of hydrolyzed anhydride polymer to produce amine reactive NHS esters. EDC reacts with a carboxyl group of the coating layer to produce an amine-reactive O-acylisourea intermediate that is susceptible to hydrolysis. The addition of NHS or sulfo-NHS stabilizes the amine-reactive O-acylisourea intermediate by converting it to an amine reactive NHS or sulfo-NHS ester, allowing for a two step procedure. Following activation of the coating, the polypeptide can then be added and the terminal amine of the polypeptide can react with the amine reactive ester to form a stable amide bond, thus conjugating the polypeptide to the coating. When EDC/NHS is employed to conjugate a polypeptide to the coating, the N-terminal amino acid can preferably be an amine containing amino acid such as lysine, ornithine, diaminobutyric acid, or diaminoproprionic acid. Any acceptable nucleophile can be employed, such as hydroxylamines, hydrazines, hydroxyls, and like amines.

EDC/NHS reactions can result in a zero length crosslinking of polypeptide to microcarrier. Linkers or spacers, such as poly(ethylene glycol) linkers (e.g., available from Quanta BioDesign, Ltd.) with a terminal amine can be added to the N-terminal amino acid of polypeptide. When adding a linker to the N-terminal amino acid, the linker is preferably a N-PG-amido-PEGₓ-acid where PG is a protecting group such as a Fmoc group, a BOC group, a CBZ group, or any other group amenable to peptide synthesis and x can be 2, 4, 6, 8, 12, 24, or any other available PEG.

In embodiments, a 1 micromolar to 10 millimolar polypeptide fluid composition, such as a solution, suspension, or like formulation, is contacted with an activated coated microcarrier to conjugate the polypeptide. For example, the polypeptide concentration can be between about 100 micromolar and about 2 millimolar, between about 500 millimolar and about 1.5 mM, or about 1 mM. The volume of the polypeptide composition and the concentration can be varied to achieve a desired density of polypeptide conjugated to the micro carrier.

The peptide can be conjugated at any suitable pH. In embodiments, the peptide can be conjugated at a pH between 7.4 and 9.2. For shorter amino acid sequences (3-15 amino acid units) a pH of 9.2 is preferred. Not limited by theory, it is believed that the terminal amino groups are more reactive at pH>9. This has resulted in higher peptide densities for shorter amino sequences than if conjugated at a lower pH (e.g., pH 5) where the amine is less reactive towards the activated carboxyl.

Referring to FIG. 5, EDC/NHS activation chemistry is used to activate the hydrolyzed anhydride polymer coated microcarrier followed by conjugation of a Vitronectin-derived peptide (VN) at pH 9.2. In this example, a rhodamine labeled amine terminated peptide (TAMRA) was also added in the peptide in the mixture at 0.25% to aid in the monitoring, for developmental purposes, the peptide conjugation step. After the peptide conjugation, the microcarrier can be blocked with, for example, ethanolamine to remove any remaining NHS esters.

Referring to FIG. 6, the VN peptide can alternatively be bound directly to the non-hydrolyzed anhydride polymer coated microcarrier. This method does not require EDC/NHS activation. A similar ethanolamine blocking step can be added as illustrated in FIG. 5 to block remaining unreacted anhydride groups.

A peptide can be conjugated to the binding polymer coated microcarrier at any density, such as at a density suitable to support culture undifferentiated human embryonic stem cells or other cell types. Peptides can be conjugated to a surface at a density of between about 1 pmol per mm² and about 50 pmol per mm² of surface of the micro carrier. For example, the peptide can be present at a density of greater than 5 pmol/mm², greater than 6 pmol/mm², greater than 7 pmol/mm², greater than 8 pmol/mm², greater than 9 pmol/mm², greater than 10 pmol/mm², greater than 12 pmol/mm², greater than 15 pmol/mm², or greater than 20 pmol/mm² of the surface of the microcarrier. In cases where the coating is thick (e.g., less than 1 micrometer) some peptide can be conjugated to the subsurface making it challenging to estimate peptide density by surface area of the binding polymer coated surface. In this instance the peptide density can be conjugated at a density of about 0.01 nmol/mg to about 1 mmol/mg assuming the microcarrier bulk density is about 1.01 to about 1.10 cm²/g. Standard BCA colorimetric techniques can be used to estimate peptide density. The amount of peptide present can vary depending on the composition of the binding polymer coating, the size of the size and shape of the surface, and the nature of the peptide itself.

The density of peptide conjugated to the surface can be controlled in several ways. For example, different levels of peptide can be conjugated to the surface varying the initial concentration of the peptide challenge solution that is reacted with the surface. Alternatively, the conjugation time can be adjusted to increase or decrease the amount of peptide conjugated. Furthermore, a species that competes with the peptide for reactive sites at the surface can be used to limit the amount of peptide bound to the surface.

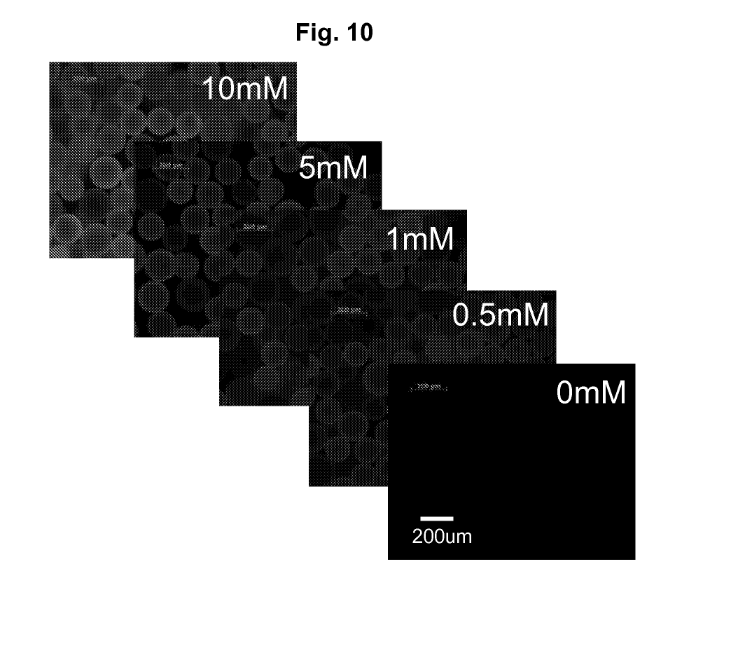

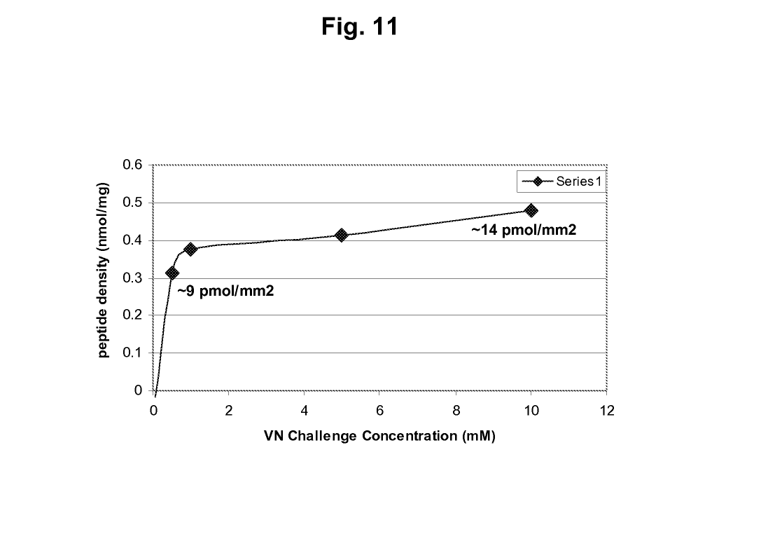

Referring to FIG. 10 and FIG. 11, a study to correlate peptide density with VN challenge concentration was carried out on microcarriers. Specifically, VN with 0.25% TAMRA labeled peptide was conjugated at concentrations of from 0.01 to 10 millimolar at pH 9. FIG. 10 shows the fluorescence intensity subtlety decrease as a function of solution concentration with 10 micromolar having the highest intensity and 0 millimolar (or no peptide) having the lowest intensity. The graph of FIG. 11 shows a clear increase in peptide density with increasing VN concentration by an on-bead bicinchoninic acid (BCA) quantitative assay (peptide densities ranged from 0.2-0.5 nanomoles per milligram or 7-15 picomoles per millimeter squared assuming a thin dEMA coating and average particle size of 180 micrometers).

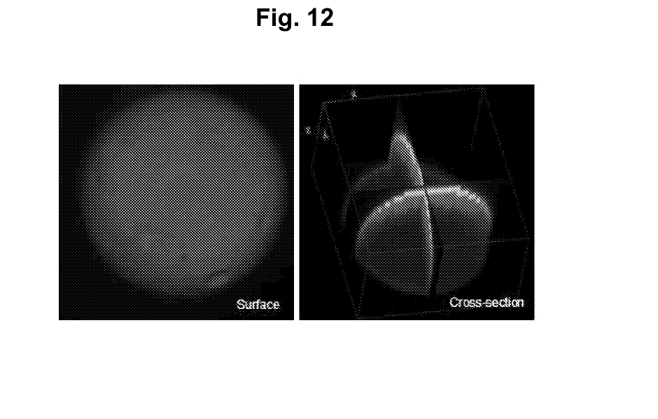

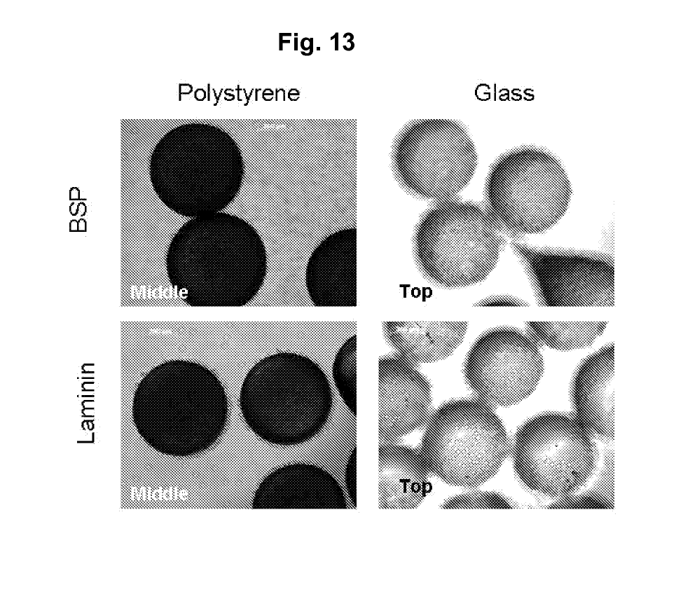

FIG. 12 shows confocal microscopy images of the bone sialoprotein (BSP)/TAMRA peptide conjugated polystyrene microcarriers. The surface image (left) shows that there is a uniform coating of peptide on the surface of the nonporous polystyrene microspheres. The cross-sectional image (right) shows that the peptide density is only at the surface of the microspheres and not in the core of the nonporous polystyrene microsphere.

The polypeptide can be cyclized or include a cyclic portion. Any suitable method for forming cyclic polypeptide can be employed. For example, an amide linkage can be created by cyclizing the free amino functionality on an appropriate amino-acid side chain and a free carboxyl group of an appropriate amino acid side chain. Also, a di-sulfide linkage can be created between free sulfhydryl groups of side chains appropriate amino acids in the peptide sequence. Any suitable technique can be employed to form cyclic polypeptides (or portions thereof). By way of example, methods described in, e.g., WO1989005150, can be employed to form cyclic polypeptides. Head-to-tail cyclic polypeptides, where the polypeptides have an amide bond between the carboxy terminus and the amino terminus can be employed. An alternative to the disulfide bond would be a di selenide bond using two selenocysteines or mixed selenide/sulfide bond, e.g., as described in Koide, et al, 1993, Chem. Pharm. Bull., 41(3):502-6; Koide, et al., 1993, Chem. Pharm. Bull., 41(9):1596-1600; or Besse and Moroder, 1997, Journal of Peptide Science, vol. 3, 442-453.

Polypeptides can be synthesized as known in the art (or alternatively produced through molecular biological techniques) or obtained from a commercial vendor, such as American Peptide Company, CEM Corporation, or GenScript Corporation. Linkers can be synthesized as known in the art or obtained from a commercial vendor, such as discrete polyethylene glycol (dPEG) linkers available from Quanta BioDesign, Ltd. Alternatively, polypeptides can be synthesized directly on the surface of the microcarrier support using standard Fmoc/Boc peptide synthesis protocols.

An example of a polypolypeptide that can be conjugated to a microcarrier is a polypeptide that includes KGGNGEPRGDTYRAY (SEQ ID NO:1), which is an RGD-containing sequence from bone sialoprotein with an additional “KGG” sequence added to the N-terminus. The lysine (K) serves as a suitable nucleophile for chemical conjugation, and the two glycine amino acids (GG) serve as spacers. Cystine (C), or another suitable amino acid, can alternatively be used for chemical conjugation, depending on the conjugation method employed. A conjugation or spacer sequence (e.g., KGG or CGG) can be present or absent. Additional examples of suitable polypeptides for conjugation with microcarriers (with or without conjugation or spacer sequences) are polypeptides that include NGEPRGDTYRAY, (SEQ ID NO:2), GRGDSPK (SEQ ID NO:3) (short fibronectin) AVTGRGDSPASS (SEQ ID NO:4) (long FN), PQVTRGDVFTMP (SEQ ID NO:5) (vitronectin), RNIAEIIKDI (SEQ ID NO:6) (lamininβ1), KYGRKRLQVQLSIRT (SEQ ID NO:7) (mLMα1 res 2719-2730), NGEPRGDTRAY (SEQ ID NO:8) (BSP-Y), NGEPRGDTYRAY (SEQ ID NO:9) (BSP), KYGAASIKVAVSADR (SEQ ID NO:10) (mLMα1 res2122-2132), KYGKAFDITYVRLKF (SEQ ID NO:11) (mLMγ1 res 139-150), KYGSETTVKYIFRLHE (SEQ ID NO:12) (mLMγ1 res 615-627), KYGTDIRVTLNRLNTF (SEQ ID NO:13) (mLMγ1 res 245-257), TSIKIRGTYSER (SEQ ID NO:14) (mLMγ1 res 650-261), TWYKIAFQRNRK (SEQ ID NO:15) (mLMα1 res 2370-2381), SINNNRWHSIYITRFGNMGS (SEQ ID NO:16) (mLMα1 res 2179-2198), KYGLALERKDHSG (SEQ ID NO:17) (tsp1 RES 87-96), or GQKCIVQTTSWSQCSKS (SEQ ID NO:18) (Cyr61 res 224-240).

In embodiments, the peptide comprises KGGK⁴DGEPRGDTYRATD¹⁷ (SEQ ID NO:19), where Lys⁴ and Asp¹⁷ together form an amide bond to cyclize a portion of the polypeptide; KGGL⁴EPRGDTYRD¹³ (SEQ ID NO:20), where Lys⁴ and Asp¹³ together form an amide bond to cyclize a portion of the polypeptide; KGGC⁴NGEPRGDTYRATC¹⁷ (SEQ ID NO:21), where Cys⁴ and Cys¹⁷ together form a disulfide bond to cyclize a portion of the polypeptide; KGGC⁴EPRGDTYRC¹³ (SEQ ID NO:22), where Cys⁴ and Cys¹³ together form a disulfide bond to cyclize a portion of the polypeptide, or KGGAVTGDGNSPASS (SEQ ID NO:23).

In embodiments, the peptide can be acetylated, amidated, or both. While these examples are provided, any peptide or peptide sequence can be conjugated to the disclosed surface.

In embodiments, the peptide polymer surface composition can contain multiple peptide sequences. These sequences can be directed toward the adhesion of either a single cell type or to enable multiple cell types to adhere to the same surface.

For any of the disclosed peptides, a conservative amino acid can be substituted for a specifically identified or known amino acid. A “conservative amino acid” refers to an amino acid that is functionally similar to a second amino acid. Such amino acids can be substituted for each other in a peptide with a minimal disturbance to the structure or function of the peptide according to well known techniques. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q).

A linker or spacer, such as a repeating poly(ethylene glycol) linker or any other suitable linker, can be used to increase distance from peptide to surface of the binding polymer coated substrate. The linker can be of any suitable length. For example, if the linker is a repeating poly(ethylene glycol) linker, the linker can contain between 2 and 10 repeating ethylene glycol units. In embodiments, the linker can be a repeating poly(ethylene glycol) linker having about 4 repeating ethylene glycol units. All, some, or none of the peptides can be conjugated to a coated microcarrier via linkers. Other potential linkers that can be employed include peptide linkers such as poly(glycine) or poly(β-alanine). Any conjugation techniques can be employed to conjugate a linker to the peptide. In embodiments, amino acids themselves can serve as linkers or spacers. For example, additional amino acids can be inserted at the N- or C-terminus of a peptide to serve as a linker or spacer. In embodiments, the linker includes polylysine, where the linker includes between 1 and 10 repeating lysine units; e.g. between 1 and 4 repeating lysine units.

### 8. Cell Culture on Peptide-Conjugated Surface-Modified Microcarriers

The disclosed microcarriers can be used in any suitable cell culture system. Typically microcarriers and cell culture media are placed in a suitable cell culture article and the microcarriers are stirred or mixed in the media. Suitable cell culture articles include bioreactors, such as the WAVE BIOREACTOR® (Invitrogen), single and multi-well plates, such as 6, 12, 96, 384, and 1536 well plates, jars, petri dishes, flasks, multi-layered flasks, beakers, plates, roller bottles, tubes, bags, membranes, cups, spinner bottles, perfusion chambers, bioreactors, CellSTACK® culture chambers (Corning, Inc.) and fermenters.

A cell culture article housing culture media containing a disclosed microcarrier can be seeded with cells. The microcarrier can be selected based on the type of cell being cultured. The cells can be of any cell type. For example, the cells can be connective tissue cells, epithelial cells, endothelial cells, hepatocytes, skeletal or smooth muscle cells, heart muscle cells, intestinal cells, kidney cells, or cells from other organs, stem cells, islet cells, blood vessel cells, lymphocytes, cancer cells, primary cells, cell lines, or like cells. The cells can be mammalian cells, preferably human cells, but can also be non-mammalian cells such as bacterial, yeast, or plant cells.

In embodiments, the cells can be stem cells which, as generally understood in the art, refer to cells that have the ability to continuously divide (self-renewal) and that are capable of differentiating into a diverse range of specialized cells. In embodiments, the stem cells are multipotent, totipotent, or pluripotent stem cells that can be isolated from an organ or tissue of a subject. Such cells are capable of giving rise to a fully differentiated or mature cell types. A stem cell can be a bone marrow-derived stem cell, autologous or otherwise, a neuronal stem cell, or an embryonic stem cell. A stem cell can be nestin positive. A stem cell can be a hematopoietic stem cell. A stem cell can be a multi-lineage cell derived from epithelial and adipose tissues, umbilical cord blood, liver, brain or other organ. In embodiments, the stem cells are pluripotent stem cells, such as pluripotent embryonic stem cells isolated from a mammal. Suitable mammals can include rodents such as mice or rats, primates including human and non-human primates. In embodiments, the microcarrier with conjugated polypeptide supports undifferentiated culture of embryonic stem cells for 5 or more passages, 7 or more passages, or 10 or more passages. Typically stems cells are passaged to a new surface after they reach about 75% confluency. The time for cells to reach 75% confluency is dependent on media, seeding density and other factors as know to those in the art.

Because human embryonic stem cells (hESC) have the ability to grown continually in culture in an undifferentiated state, the hESC for use with the disclosed microcarriers can be obtained from an established cell line. Examples of human embryonic stem cell lines that have been established include, for example, BG01V/hOG cells (available from Invitrogen and described herein), H1, H7, H9, H13 or H14 (available from WiCell; U. Wisconsin) (Thompson (1998) Science, 282:1145); hESBGN-01, hESBGN-02, hESBGN-03 (BresaGen, Inc., Athens, Ga.); HES-1, HES-2, HES-3, HES-4, HES-5, HES-6 (from ES Cell International, Inc., Singapore); HSF-1, HSF-6 (from University of California at San Francisco); I 3, I 3.2, I 3.3, I 4, I 6, I 6.2, J 3, J 3.2 (derived at the Technion-Israel Institute of Technology, Haifa, Israel); UCSF-1 and UCSF-2 (Genbacev et al., Fertil. Steril., 83(5):1517-29, 2005); lines HUES 1-17 (Cowan et al., NEJM 350(13):1353-56, 2004); and line ACT-14 (Klimanskaya et al., Lancet, 365(9471):1636-41, 2005). Embryonic stem cells can also be obtained directly from primary embryonic tissue. Typically this is done using frozen in vitro fertilized eggs at the blastocyst stage, which would otherwise be discarded.

Other sources of pluripotent stem cells include induced primate pluripotent stem (iPS) cells. iPS cells refer to cells, obtained from a juvenile or adult mammal, such as a human, that are genetically modified, e.g., by transfection with one or more appropriate vectors, such that they are reprogrammed to attain the phenotype of a pluripotent stem cell such as an hESC. Phenotypic traits attained by these reprogrammed cells include morphology resembling stem cells isolated from a blastocyst and surface antigen expression, gene expression and telomerase activity resembling blastocyst derived embryonic stem cells. The iPS cells typically have the ability to differentiate into at least one cell type from each of the primary germ layers: ectoderm, endoderm and mesoderm. The iPS cells, like hESC, also form teratomas when injected into immuno-deficient mice, e.g., SCID mice. (Takahashi, et al., (2007) Cell 131(5):861; Yu et al., (2007) Science 318:5858).

To maintain stem cells in an undifferentiated state it can be desirable to minimize non-specific interaction or attachment of the cells with the surface of the microcarrier, while obtaining selective attachment to the polypeptide(s) attached to the surface. The ability of stem cells to attach to the surface of a microcarrier without conjugated polypeptide can be tested prior to conjugating polypeptide to determine whether the microcarrier provides for little to no non-specific interaction or attachment of stem cells. Once a suitable microcarrier has been selected, cells can be seeded in culture medium containing the microcarriers.

Prior to seeding cells, the cells, regardless or cell type, can be harvested and suspended in a suitable medium, such as a growth medium in which the cells are to be cultured once seeded. For example, the cells can be suspended in and cultured in a serum-containing medium, a conditioned medium, or a chemically-defined medium. As used herein, “chemically-defined medium” means cell culture media that contains no components of unknown composition. Chemically defined cell culture media may, in embodiments, contain no proteins, hydrozylates, or peptides of unknown composition. In some embodiments, chemically defined media contains polypeptides or proteins of known composition, such as recombinant growth hormones. Because all components of chemically-defined media have a known chemical structure, variability in culture conditions and thus variability in cell response can be reduced, to increase reproducibility. In addition, the possibility of contamination is reduced. Further, the ability to scale up is made easier due, at least in part, to the factors discussed above. Chemically defined cell culture media are commercially available from, for example, Invitrogen (Carlsbad, Calif.) as STEMPRO®, a fully serum- and feeder-free (SFM) specially formulated from the growth and expansion of embryonic stem cells, and Xvivo (Lonza), and Stem Cell Technologies, Inc. as mTeSR™ 1 maintenance media for human embryonic stem cells.

One or more growth or other factors can be added to the medium in which cells are incubated with the microcarriers conjugated to polypeptide. The factors can facilitate cellular proliferation, adhesion, self-renewal, differentiation, or like facilitations. Factors that can be added to or included in the medium include, for example, muscle morphogenic factor (MMP), vascular endothelium growth factor (VEGF), interleukins, nerve growth factor (NGF), erythropoietin, platelet derived growth factor (PDGF), epidermal growth factor (EGF), activin A (ACT) such as activin A, hematopoietic growth factors, retinoic acid (RA), interferons, fibroblastic growth factors, such as basic fibroblast growth factor (bFGF), bone morphogenetic protein (BMP), peptide growth factors, heparin binding growth factor (HBGF), hepatocyte growth factor, tumor necrosis factors, insulin-like growth factors (IGF) I and II, transforming growth factors, such as transforming growth factor-β1 (TGFβ1), and colony stimulating factors.

The cells can be seeded at any suitable concentration. Typically, the cells are seeded at about 10,000 cells/cm² of microcarrier to about 500,000 cells/cm². For example, cells can be seeded at about 50,000 cells/cm² of substrate to about 150,000 cells/cm². However, higher and lower concentrations can be selected. The incubation time and conditions, such as temperature, CO₂ and O₂ levels, growth medium, and like considerations, will depend on the nature of the cells being cultured and can be readily modified. The amount of time that the cells are cultured with the microcarriers can vary depending on the cell response desired.

The cultured cells can be used for any suitable purpose, including: i) obtaining sufficient amounts of undifferentiated stem cells cultured on a synthetic surface in a chemically defined medium for use in investigational studies or for developing therapeutic uses; ii) for investigational studies of the cells in culture; iii) for developing therapeutic uses; iv) for therapeutic purposes; v) for studying gene expression, e.g., by creating cDNA libraries; vi) for studying drug and toxicity screening; and like purposes, or combinations thereof.

One suitable way to determine whether cells are undifferentiated is to determine the presence of the OCT4 marker. In embodiments, the undifferentiated stems cells cultured on the disclosed microcarriers for 5, 7, or 10 or more passages retain the ability to be differentiated.

FIG. 13 illustrates HT1080 cell adhesion on bone sialoprotein peptide (BSP) derived polystyrene and glass microspheres prepared using the disclosed methods. Microscopic images show that the disclosed microcarriers support short term (1 hour) adhesion and spreading of HT1080 cells. Laminin coated microspheres were used as a positive control for cell attachment and spreading. Cell attachment and spreading on BSP-derivatized beads were comparable to the laminin coated beads.

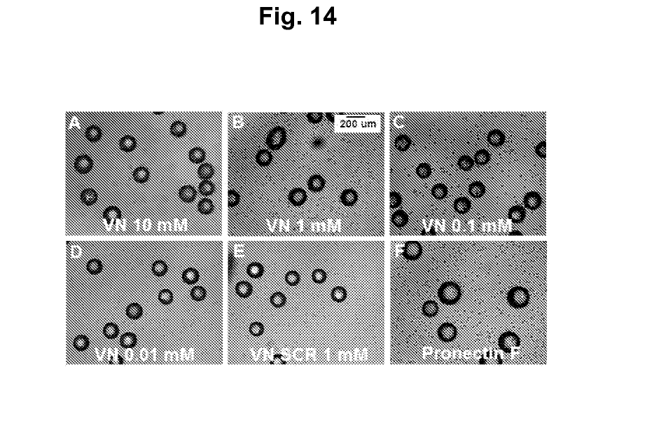

FIG. 14 shows HT1080 cell adhesion on glass-VN-peptide microcarriers prepared using the disclosed method. The microcarriers were prepared with different levels of VN peptide and used to study the relationship between microcarrier peptide density and HT1080 cell adhesion. Cell attachment was assessed using an inverted light microscope. Glass microcarriers with VN conjugated at 10, 1, and 0.1 millimolar (images A, B, and C) provided similar short term adhesion of HT1080 cells as the positive control Pronectin® F (polystyrene grafted with recombinant fibronectin, commercially available from Sigma-SoloHill, image F). BCA peptide density quantification of the 10, 1, and 0.1 millimolar VN samples were 14.1, 11.4 and 8.3 picomoles per millimeter squared, respectively. An obvious drop in HT1080 short term adhesion was observed at 0.01 millimolar VN (<7 picomoles per millimeter squared) (image D).

FIG. 15 shows images of BG01V/hOG human embryonic stem cell growth on Vitronectin peptide-modified glass microcarriers 5 days after seeding where A is the brightfield image, and B is the fluorescence image (FITC).

FIG. 16 graphs the quantification of BG01V/hOG cells after 2 days and 5 days culture performed on Vitronectin peptide-modified glass microcarriers (LDG-dEMA-VN), on Matrigel coated beads (Matrigel™ CM) and Cytodex™ 3 as comparative example. The graph shows the advantage provided by the disclosed microcarriers after 5 days culture over the known collagen coated microcarrier. Furthermore, the graph shows that the disclosed microcarrier beads performed comparable to a known industry standard Matrigel coated beads.

In embodiments, the disclosure provides microspheres having a maleic anhydride polymer coating, which microspheres are particularly useful for at least the following reasons.

The microcarriers enable an activated surface that a VN, BSP peptide, and related RGD peptides, and like peptides can be directly conjugated to and used for microcarrier suspension cell culture.

The direct binding procedure does not require “pre-activation” and avoids the use of EDC/NHS chemistry.

The hydrolyzed anhydride polymer coated microcarriers can be regenerated and subsequently used for direct conjugation of peptides.

The EDC/NHS “indirect” procedure can optionally be used on hydrolyzed anhydride polymer coated micro carriers.

If desired, the reactive anhydride polymer coated microcarriers can be used to covalently attach animal sourced matrices such as Matrigel™, collagen, and like extra-cellular matrices.

The disclosed anhydride polymer coated microcarriers can be chemically modified (i.e., derivatized) with, for example, charged, hydrophilic, hydrophobic, and like residues to enhance protein binding, and without introducing negative charge as with dEMA, for example, accomplishing EDC/NHS activation followed by ethanolamine blocking

The disclosed microcarriers can undergo and survive sterilization protocols (e.g., autoclaving, irradiation) prior to peptide conjugation.

The anhydride binding polymer can be conveniently applied, such as by dip coating directly on an amine functionalized or like nucleophilic microcarrier.

The peptide-modified microcarrier can overcome many limitations of animal derived Matrigel® and Collagen, such as minimizing lot-to-lot variability.

Biospecific specific attachment of the cells to the microcarriers in known media (e.g., serum-free culture) can be accomplished.

Unlike collagen and Matrigel™ coated substrates, the disclosed peptide-surface modified microcarriers are stable and do not require specific storage conditions (cf. collagen is stored at 4° C. and Matrigel™ at −20° C.). This provides off-the-shelf ease of use and convenience.

The disclosed peptide-modified microcarriers can be used with a wide variety of other cell lines.

The disclosed surface treatment process can be applied to various substrates such as glass beads, fiber, or like high surface area substrates.

### EXAMPLES

The following examples serve to more fully describe the manner of using the above-described disclosure, and to further set forth the best modes contemplated for carrying out various aspects of the disclosure. It is understood that these examples do not limit the scope of this disclosure, but rather are presented for illustrative purposes. The working examples further describe the methods and how to make the articles disclosed peptide-modified microcarriers and there use in cell culture.

### Example 1

Coating of anhydride polymer onto glass beads. To a 50 microliters polypropylene centrifuge tube was added 1,000 milligrams of dry low density glass microcarrier beads (Sigma, about 150 to 210 micrometers, 1.03 g/cc) and 15 milliliters of 25% aminopropylsilsesquioxane (APS) in water. The bead slurry was mixed on an orbital shaker for 3 minutes. The beads were spun down by centrifugation (4,000 RCF, 5 minutes) and the APS solution was removed. The beads were then aspiration washed with DI water and ethanol (3×40 milliliters each) using centrifugation. After the final ethanol wash, the APS coated glass beads were vacuum dried overnight. The ninhydrin test verified the presence of primary amine functionality. Following, to 500 milligrams of the APS coated glass beads was transferred to a 15 milliliters polypropylene centrifuge tube and to the beads was added 10 milliliters of dEMA (2 milligrams per milliliter in NMP/IPA 1:4). The slurry was mixed on an orbiter shaker for 10 minutes. The beads were aspiration washed with NMP, water, and ethanol (3×5 milliliters each) and air dried in a vacuum oven. Crystal violet staining of hydrolyzed anhydride groups verified the presence of the dEMA coating as shown in FIG. 9.

### Example 2

Conjugation of peptide to anhydride polymer coated microcarriers. 50 milligrams of dry, hydrolyzed anhydride polymer coated glass beads (about 150 to 210 micrometer particle size) was transferred to a 2 milliliter centrifuge tube. A 1 milliliter solution of EDC/NHS (200/50 millimolar in DI water) was added to the beads and allowed to mix on an orbital shaker for 60 minutes. The solution was aspirated, rinsed twice with 1 milliliter of water, aspirated, and then 1 milliliter of Vitronectin (Ac-KGGPQVTRGDVFTMP-NH2, SEQ ID NO:5) or Vitronectin RGD scrambled (Ac-KGGPQVTGRDVFTMP-NH2, SEQ ID NO:24) from American Peptide Company), scrambled (0-10 millimolar in borate buffer, pH 9.2, spiked with 0.25% Rhodamine peptide ((5/6TAMRA-Gly-Arg-Gly-Asp-Ser-Pro-Ile-Ile-Lys-NH₂(SEQ ID NO:25)—product #347678) was added and allowed mix for 60 min. The peptide solution was removed by aspiration and the beads were treated with 1.5 milliliters of 1M ethanolamine pH 8 for 30 minutes followed by washing with PBS (1.5 milliliter×5), 1% SDS (1×1.5 milliliter×1.5 minutes), and DI Water and ethanol (1.5 milliliter×5) and dried under a gentle stream of nitrogen.

### Example 3

Crystal violet staining to verify coating of microbeads. Crystal violet staining of base microbeads and coated microbeads was performed to verify that the polymer layer was coated grafted to the beads. Small samples of the dry microbeads were placed in a 2 milliliter centrifuge tube. 500 microliters of a 1:5 dilution of crystal violet blue in water was added to the centrifuge tube. After 5 minutes, the sample was aspiration washed with DI water or until top solution was clear and colorless. Staining of the microspheres was assessed using a light microscope and representative images are presented in FIG. 9. The hydrolyzed anhydride polymer coated microspheres were uniformly stained, while the uncoated microspheres were un-stained.

### Example 4

Peptide Density Estimation. The density of polypeptide conjugated to the coated microcarrier was estimated by an Interchem (Montiucon Codex, France) bicinchoninic acid (BCA) assay. The BCA working reagent was prepared by adding 1 part of reagent B to 50 parts of reagent A in a 50 mL centrifuge tube. The standard solutions were prepared by serial dilution of a 10 mM Vitronectin solution down to 1 micromolar. 10 mg of dry VN modified microcarriers were added to separate wells of a Corning ultra low attachment (ULA) 24-well plate. 25 microliters of each standard solution was also introduced into separate wells of the ULA 24-well plate. To each standard solution and sample was added 800 microliters of the BCA working reagent per test well and the plate was incubated for 2 hours at 25° C. (gently mixing the plate every 30 min to re-suspend microcarriers). 750 microliters of BCA color developed standard and sample solutions were removed (place pipette tip in corner of well to minimize transfer of beads from sample well) and the optical absorbance was read at 562 nm (instrument blanked with PBS). To estimate peptide density, the blank absorbance was subtracted from all others to get net absorbance to generate a standard curve of net absorbance as a function of VN concentration. The linear fit up to 5 mM was used to generate a correlation formula. The absorbance of the base bead (no VN) was subtracted from VN-sample absorbance to get the sample net absorbance. The correlation formula was then used to estimate peptide density in nmol/mg and pmol/mm². The results are shown in FIG. 11.

### Example 5

HT1080 cell preparation. HT1080 human fibrosarcoma cells (ATCC # CCL-121) were maintained in Iscove's Modified Dulbecco's Medium (IMDM) with 10% Fetal Bovine Serum (FBS) at 37° C., 5% CO₂. The day of the assay, cells were harvested by trypsin treatment, re-suspended in IMDM with 10% FBS, and incubated for 1 h at 37° C., 5% CO₂. After recovery, the cells were washed and re-suspended in 0.1% Bovine Serum Albumin (BSA) in IMDM.

HT1080 Cell adhesion assay on microcarriers. Cells were trypsinized and allowed to recover in Iscove's Modified Dulbecco's Medium (IMDM) with 10% Fetal Bovine Serum (FBS) for 30 minutes at 37° C., 5% CO₂. After recovery, the cells were washed and re-suspended in 0.1% Bovine Serum Albumin (BSA) in IMDM. Approximately 3 mg of vitronectin-derivatized micro carriers (Ac-KGGPQVTRGDVFTMP-NH2, SEQ ID NO:5) was transferred to a 2 mL centrifuge tube and blocked with 2 mL of 1% BSA in D-PBS for 1 hr at room temperature. The microspheres were then washed with 2 mL of D-PBS, resuspended in 200 microliters of 0.1% BSA in IMDM prior to cell seeding and placed in a 24-well Corning Ultra low attachment microplate. 200 microliters of resuspended cells were placed in each well of the 24-well Corning microplate. The bead and cell suspension was incubated for 1 hr at 37 C, 5% CO₂. The media was removed and the beads were washed in the wells with D-PBS (2×2 mL). Cellular attachment and spreading was assessed using Ziess Axiovert 200M inverted microscope. Images of the cells adhered to the microcarrier are shown in FIG. 13 and FIG. 14. When the microcarrier was conjugated with the Vitronectin RGD scrambled sequence (Ac-KGGPQVTGRDVFTMP-NH2, SEQ ID NO:24), no cells adhered to the microcarriers also as shown in FIG. 14E.

### Example 6

BG01V/hOG cell adhesion and cell expansion assay. BG01V/hOG cells (Invitrogen) were maintained on Matrigel coated TCT 75 Flask (Corning) in serum free mTERS1 medium containing 50 microg/mL Hygromycin B (STEMCELL Technologie). Daily medium changes began after the first 48 h in culture. Cells were passaged every 5 to 6 days using collagenase IV (Invitrogen) and mechanical scraping.

For the assay, aggregate colonies were harvested and resuspended in fresh mTERS1 medium. Cells were seeded to a 24-wells Corning Ultra low attachment microplate (1.5×105 cells per cm2) containing the disclosed microcarriers or Cytodex™ 3 microcarrier available from GE Healthcare as a comparative example. The volume was adjusted to 600 microliters with culture medium. Cells were allowed to attach to the microcarriers for 48 h without agitation. 2 days after seeding, cellular attachment and spreading was assessed using Ziess Axiovert 200M inverted microscope. Quantitative analysis was also performed as follows. The media was removed and the beads were washed in the wells with D-PBS (2×3 mL). The D-PBS was removed and replaced with 200 microliters of CellTiter-Glo reagent (Promega). The microplate was place in the shaker for 10 min at RT and luminescence was measured. For the cell expansion assay, the same seeding protocol was used and cells were maintained in static condition over the course of cell expansion. After 48 h cell attachment, the culture medium was changed daily after sedimentation of the cells and the beads. After 5 days, cell spreading and cell quantification was assessed using the same methods described above. The results are shown in FIG. 15.

### Example 7

Regeneration of hydrolyzed anhydride polymer coated microcarriers. Approximately 100 mg of water hydrolyzed anhydride polymer coated microspheres (washed with water, DMSO, and CH₂Cl₂) were transferred to a small glass vial and placed in a vacuum oven at 120° C. for 4 hours, then cooled to room temperature. The resulting thermally treated beads were ready for immediate suspension cell culture.

The disclosure has been described with reference to various specific embodiments and techniques. However, it should be understood that many variations and modifications are possible while remaining within the scope of the disclosure.

1. A method of making cell-culture microspheres comprising:
contacting a copolymer comprising maleic anhydride and a first monomer and amine-functionalized microspheres to provide copolymer surface-modified microspheres; and
conjugating the surface-modified microspheres with a peptide source.

2. The method of claim 1 wherein the conjugating is accomplished at a pH of about 9.

3. The method of claim 1 wherein the peptide source comprises at least one of:

| | |
|---|---|
| Ac-KGGNGEPRGDTYRAY | (SEQ ID NO: 1) (BSP), |
| Ac-KGGPQVTRGDVTMP-NH₂ | (SEQ ID NO: 26) (VN), |

or a combination thereof.

4. The method of claim 1 wherein the conjugating comprises:
hydrolyzing a portion of the maleic anhydride groups on the copolymer surface-modified microspheres;
contacting a portion of the hydrolyzed maleic anhydride groups on the EMA surface-modified microspheres and an activating agent to form an activated surface on the microspheres; and
contacting the activated surface of the microspheres with the peptide source.

5. The method of claim 4 wherein the activating agent comprises EDC/NHS or EDC/s-NHS.

6. The method of claim 1 wherein the first monomer comprises ethylene.

7. A method for cell culture comprising:
contacting cells and microspheres having a surface-modified with a maleic anhydride containing polymer, and the polymer having a conjugated peptide.

8. The method of claim 7, wherein the cells comprise stem cells, hepatocytes, neural stem cells, embryonic stem cells, and combinations thereof.

9. The method of claim 7, wherein the contacting is accomplished having a microsphere suspension in a chemically-defined cell culture medium.

10. A cell culture article prepared by the method of claim 1.

11. A cell culture article comprising a microcarrier having a peptide-modified polymer surface of the formula (I): 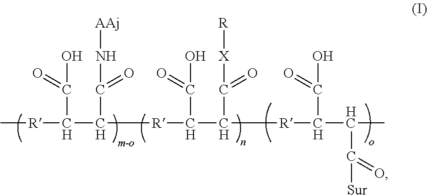
where
m-o is an integer representing the mers containing a carboxy group and an AAⱼ peptide-modified group,
n is an integer representing the mers containing a optional pre-blocked group (X—R) and a carboxy group,
o is an integer representing the mers containing a carboxy group and surface attachment group (Sur),
AAⱼ comprises at least one covalently attached peptide comprised of an AAⱼ peptide-modification source having amino acids,
j is an integer representing from 5 to 50 amino acids,
Sur comprises a surface attachment group,
X is a divalent —NH—, —NR—, —O—, or —S— of a pre-block source,
R is H, or a substituted or an unsubstituted, linear or branched, alkyl group, an oligo(ethylene oxide), an oligo(ethylene glycol), or a dialkyl amine of the pre-block source,
R′ is a substituted or an unsubstituted, linear or branched, hydrocarbylene having from 2 to about 10 carbon atoms, and
the relative mer ratio (m-o:n:o) is from about 0.5:1:0.01 to about 10:1:0.001,
and salts thereof.

12. The cell culture article of claim 11, wherein AAⱼ comprises at least one peptide source selected from:

| | |
|---|---|
| Ac-KGGNGEPRGDTYRAY | (SEQ ID NO: 1) (BSP), |
| Ac-KGGPQVTRGDVIMP-NH₂ | (SEQ ID NO: 26) (VN), |

or a combination thereof.

13. The cell culture article of claim 11 wherein the pre-block agent or pre-block source comprises an alkyl amine, an alkylhydroxy amine, an alkoxyalkyl amine, an alcohol, an alkyl thiol, water, or H₂S.

14. A method for regenerating the activity of microcarrier having a surface comprising hydrolyzed maleic anhydride groups comprising:
heating the microcarrier in a vacuum.

15. The method of claim 14 wherein the heating is at about 120° C. for about 4 hrs.

16. The method according to claim 14, wherein the microcarrier surface further comprises a peptide conjugated to the surface.


SEQUENCE LISTING (26 sequences)

SEQ ID NO: 1 - protein, 15 aa, Artificial Sequence
  Synthetic polypeptide
KGGNGEPRGDTYRAY

SEQ ID NO: 2 - protein, 12 aa, Artificial Sequence
  Synthetic polypeptide
NGEPRGDTYRAY

SEQ ID NO: 3 - protein, 7 aa, Artificial Sequence
  Synthetic polypeptide
GRGDSPK

SEQ ID NO: 4 - protein, 12 aa, Artificial Sequence
  Synthetic polypeptide
AVTGRGDSPASS

SEQ ID NO: 5 - protein, 12 aa, Artificial Sequence
  Synthetic polypeptide
PQVTRGDVFTMP

SEQ ID NO: 6 - protein, 10 aa, Artificial Sequence
  Synthetic polypeptide
RNIAEIIKDI

SEQ ID NO: 7 - protein, 15 aa, Artificial Sequence
  Synthetic polypeptide
KYGRKRLQVQLSIRT

SEQ ID NO: 8 - protein, 11 aa, Artificial Sequence
  Synthetic polypeptide
NGEPRGDTRAY

SEQ ID NO: 9 - protein, 12 aa, Artificial Sequence
  Synthetic polypeptide
NGEPRGDTYRAY

SEQ ID NO: 10 - protein, 15 aa, Artificial Sequence
  Synthetic polypeptide
KYGAASIKVAVSADR

SEQ ID NO: 11 - protein, 15 aa, Artificial Sequence
  Synthetic polypeptide
KYGKAFDITYVRLKF

SEQ ID NO: 12 - protein, 16 aa, Artificial Sequence
  Synthetic polypeptide
KYGSETTVKYIFRLHE

SEQ ID NO: 13 - protein, 16 aa, Artificial Sequence
  Synthetic polypeptide
KYGTDIRVTLNRLNTF

SEQ ID NO: 14 - protein, 12 aa, Artificial Sequence
  Synthetic polypeptide
TSIKIRGTYSER

SEQ ID NO: 15 - protein, 12 aa, Artificial Sequence
  Synthetic polypeptide
TWYKIAFQRNRK

SEQ ID NO: 16 - protein, 20 aa, Artificial Sequence
  Synthetic polypeptide
SINNNRWHSIYITRFGNMGS

SEQ ID NO: 17 - protein, 13 aa, Artificial Sequence
  Synthetic polypeptide
KYGLALERKDHSG

SEQ ID NO: 18 - protein, 17 aa, Artificial Sequence
  Synthetic polypeptide
GQKCIVQTTSWSQCSKS

SEQ ID NO: 19 - protein, 17 aa, Artificial Sequence
  Synthetic polypeptide
KGGKDGEPRGDTYRATD

SEQ ID NO: 20 - protein, 13 aa, Artificial Sequence
  Synthetic polypeptide
KGGLEPRGDTYRD

SEQ ID NO: 21 - protein, 17 aa, Artificial Sequence
  Synthetic polypeptide
KGGCNGEPRGDTYRATC

SEQ ID NO: 22 - protein, 13 aa, Artificial Sequence
  Synthetic polypeptide
KGGCEPRGDTYRC

SEQ ID NO: 23 - protein, 15 aa, Artificial Sequence
  Synthetic polypeptide
KGGAVTGDGNSPASS

SEQ ID NO: 24 - protein, 15 aa, Artificial Sequence
  Synthetic polypeptide
KGGPQVTRGDVFTMP

SEQ ID NO: 25 - protein, 9 aa, Artificial Sequence
  Synthetic polypeptide
GRGESPIIK

SEQ ID NO: 26 - protein, 14 aa, Artificial Sequence
  Synthetic polypeptide
KGGPQVTRGDVTMP